(19) United States
(12) Patent Application Publication
(10) Pub. No.: US 2018/0312744 A1
(43) Pub. Date: Nov. 1, 2018

(54) GENES ENCODING CELLULASE FOR HYDROLYZING GUAR FRACTURING FLUIDS UNDER EXTREME WELL CONDITIONS

(71) Applicant: BASF Enzymes, LLC, San Diego, CA (US)

(72) Inventors: Xuqiu Tan, San Diego, CA (US); Kenneth E. Barrett, San Diego, CA (US); Adrienne Huston Davenport, San Diego, CA (US); Lawrence E. Whipple, San Diego, CA (US); Hugo D. Urbina, San Diego, CA (US); Bin Zhang, San Diego, CA (US); Mark A. Wall, San Diego, CA (US)

(21) Appl. No.: 15/835,386

(22) Filed: Dec. 7, 2017

Related U.S. Application Data
(63) Continuation of application No. 14/389,065, filed on Sep. 29, 2014, now abandoned.
(60) Provisional application No. 61/618,610, filed on Mar. 30, 2012.
(60) Provisional application No. 61/660,556, filed on Jun. 15, 2012.

(51) Int. Cl. C09K 8/68; C12P 19/04; C12N 9/42; C12P 19/14

(52) U.S. Cl. CPC C09K 8/68; C09K 2208/24; C12P 19/14; C12N 9/2437; C12P 19/04

(57) ABSTRACT

Polynucleotide sequences encoding a thermostable cellulase and directing its increased expression are provided, and hydraulic fracturing compositions comprising such thermostable cellulase.

### RELATED APPLICATIONS

This application claims the benefit of priority under 35 U.S.C. § 119(e) to U.S. Provisional Applications 61/618,610, filed Mar. 30, 2012 and 61/660,556, filed Jun. 15, 2012 each of which is herein incorporated by reference in its entirety.

### FIELD OF THE INVENTION

Polynucleotide sequences encoding a cellulase are provided. In particular, the provided sequences may provide increased expression of a specific, thermostable, thermotolerant, pressure stable cellulase, such as a cellulase for hydrolyzing guar fracturing fluids under extreme well conditions.

### SEQUENCE LISTING

This application is being filed electronically via the USPTO EFS-WEB server, as authorized and set forth in MPEP § 502.05 and this electronic filing includes an electronically submitted sequence listing; the entire content of this sequence listing is hereby incorporated by reference into the specification of this application. The sequence listing is identified on the electronically filed ASCII (.txt) text file as follows:

| File Name | Date of Creation | Size |
|---|---|---|
| D2570_SEQLISTING | Mar. 9, 2013 | 40.4 kb |

### BACKGROUND

O-Glycosyl hydrolases (EC 3.2.1.-) are a widespread group of naturally-occurring enzymes that hydrolyze the glycosidic bond between two or more carbohydrates or between a carbohydrate and a non-carbohydrate moiety. The International Union of Biochemistry and Molecular Biology (IUBMB) enzyme nomenclature of glycosyl hydrolases (or glycosylases) is based principally on their substrate specificity and occasionally on their molecular mechanism (Nomenclature Committee of the International Union of Biochemistry and Molecular Biology (NC-IUBMB), Accessed Oct. 24, 2011).

IUBMB Enzyme Nomenclature EC 3.2.1.4 has been designated for a subgroup group of glycosylase-type enzymes termed “cellulases.” Other names used for enzymes belonging to this group include: endoglucanase, endo-1,4-beta-glucanase, carboxymethyl cellulase, and beta-1,4-glucanase. The reaction catalyzed by enzymes belonging to this group is the endo-hydrolysis of 1,4-beta-D-glycosidic linkages in cellulose, lichenin, and cereal beta-D-glucans (such as barley beta-glucan). Since the predominant activities of the disclosed cellulase of the present invention are the endo-hydrolysis of barley beta-glucan and carboxymethyl cellulose, it is appropriately ascribed the IUBMB Enzyme Nomenclature EC 3.2.1.4.

An alternative classification of glycosyl hydrolases is based on amino acid sequence similarities (Henrissat, B. Accessed at UniProt Oct. 26, 2011). According to this classification scheme, glycosyl hydrolases can be divided into more than 70 families. Based on a comparison of the primary amino acid sequence of the disclosed cellulase of the present invention with the sequences of other glycosyl hydrolases contained in public databases, the disclosed cellulase of the present invention may be assigned to glycosyl hydrolase Family 5. This family contains more than 20 endoglucanases (IUBMB Enzyme Nomenclature EC 3.2.1.4) whose predominant catalytic activity is the endo-hydrolysis of beta-1,4-glycosidic linkages in cellulosic substrates. Using this second way of classifying enzymes provides further support for the conclusion that the disclosed cellulase of the present invention should be ascribed the IUBMB Enzyme Nomenclature EC 3.2.1.4.

Cellulases are used for a variety of industrial and commercial purposes including but not limited to, oil and gas exploration, food and beverage, alcohol production potable or fuel, e.g. brewing, ethanol, wine, flavor, fragrance, textile, detergents, paper, pulp, environmental, and agriculture, as well as in research purposes (Rebecca S. Bryant, Erie C. Donaldson, Teh Fu Yen, George V. Chilingarian, Chapter 14 Microbial Enhanced Oil Recovery, In: Erle C. Donaldson. George V. Chilingarian and Teh Fu Yen, Editor(s), Developments in Petroleum Science, Elsevier, 1989, Volume 17. Part B, Pages 423-450; M. Karmakar and R. R. Ray, 2011. Current Trends in Research and Application of Microbial Cellulases. Research Journal of Microbiology, 6: 41-53).

Enzyme breakers have been successfully used in water based fracturing fluids since the early 1990s to hydrolyze polymeric viscosifiers (Brannon H D, Tjon-Joe-Pin R M. Carman P S, Wood W D, “Enzyme breaker technology: a decade of improved well stimulation”, paper SPE 84213 presented at the SPE Annual Technical Conference and Exhibition in Denver, Colo., USA, 5-8 Oct. 2003.

### SUMMARY

Enzymes are proteins with 3-dimensional structures that are sensitive to higher temperatures and higher alkaline conditions present in many fracturing operations. The identification and/or engineering of enzymes that retain catalytic activity under challenging down-hole conditions (e.g., high temperature and pH) would broaden enzyme breaker applications in water based fracturing.

A composition is disclosed comprising a polymeric viscosifier, a surfactant, a thermostabilizer, and an enzyme breaker comprising a wild-type cellulase derived from a hyperthermophilic bacterium or a mutated variant thereof.

In some embodiments, the viscosifier is a guar gel comprising a linear guar, a crosslinked guar, or mixtures thereof. In some embodiments, the enzyme breaker specifically hydrolyzes β-1,4 glycosidic bonds in the guar gel. In some embodiments, the enzyme breaker does not specifically hydrolyze α-1,6 glycosidic bonds in the guar gel. In some embodiments, the enzyme breaker retains its ability to hydrolyze β-1,4 glycosidic bonds in the guar gel at temperatures up to about 275° F. In some embodiments, the enzyme breaker retains its ability to hydrolyze β-1,4 glycosidic bonds in the guar gel at a pH of up to about 11.

In some embodiments, the enzyme breaker in the composition is a mutated variant comprising 12 mutations relative to the wild type cellulase. The 12 mutations may be selected from the group consisting of F38Y, Y61Q, M69E, D70P, R71 S, 194Q, I166V, S183R, S191A, E212P, L231V, M276A, E277S, R280G, T297P, and T301Q. In some embodiments, the enzyme breaker has SEQ ID. NO. 2. In some embodiments, the enzyme breaker is encoded by a polynucleotide having SEQ ID. NO. 1. In some embodiments, the enzyme breaker is a mutated variant of the wild-type cellulase, and has a melting temperature that is at least 20° F. greater than the melting temperature of the wild type cellulase at about pH 6.5, and at least 10° F. greater than the melting temperature of the wild type cellulase at about pH 10.5.

A method is disclosed for reducing the viscosity of a polysaccharide gel that includes β-1,4 glycosidic bonds. The method includes contacting the polysaccharide gel with a cellulase variant under permissive conditions and for a period of time sufficient to allow the cellulase to hydrolyze the β-1,4 glycosidic bonds in the polysaccharide gel thereby reducing the viscosity of the guar gel. The cellulase variant comprises at least 12 mutations compared to a wild-type cellulase derived from a hyperthermophilic bacterium, and the cellulase variant exhibits increased temperature and pH tolerance compared to the wild-type cellulase.

In some embodiments of the method, the at least 12 mutations in the cellulase variant were generated by Gene Site Saturation Mutagenesis comprising repeated cycles of reductive reassortment, recombination, and selection.

In some embodiments of the method, the polysaccharide gel comprises a linear guar, a crosslinked guar, or mixtures thereof.

In some embodiments of the method, the cellulase variant has SEQ ID NO:2. In some embodiments of the method, the cellulase variant is encoded by a polynucleotide having SEQ ID. NO. 1.

In some embodiments of the method, permissive conditions comprise a temperature range of from about 180° F. to about 275° F. In some embodiments of the method, permissive conditions comprise a pH range of from about 9 to about 11.

A method of treating a subterranean formation is also disclosed. The method comprises: introducing into the subterranean formation a fracturing fluid that comprises a polysaccharide gel and a cellulase variant; and allowing the gel and the cellulase variant to react under permissive conditions and for a period of time sufficient to allow the cellulase variant to hydrolyze at least some of the β-1,4 glycosidic bonds, but not the α-1,6 glycosidic bonds, in the gel, thereby generating a reduced viscosity fracturing fluid. The cellulase variant may comprise at least 12 mutations compared to a parental wild-type cellulase derived from a hyperthermophilic bacterium, and the cellulase variant may exhibit increased temperature and pH tolerance compared to the wild-type cellulase.

In some embodiments of the method, the reduced viscosity fracturing fluid generated by the cellulase variant comprises less residue that a comparable reduced viscosity fracturing fluid generated by a chemical breaker, wherein the dosage of cellulase variant and chemical breaker provide substantially the same reduction in viscosity.

In some embodiments of the method, the reduced viscosity fracturing fluid generated by the cellulase variant retains greater conductivity than a comparable reduced viscosity fracturing fluid generated by a chemical breaker, wherein the dosage of cellulase variant and chemical breaker provide substantially the same reduction in viscosity.

In another embodiment the invention comprises SEQ ID NO:1, wherein said sequence encodes a protein. In a further embodiment the invention comprises a nucleotide sequence encoding a cellulase derived from Thermotoga maritima, or SEQ ID NO:3, comprising at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, or any combination thereof, wherein optionally, any such mutations are silent. In a further embodiment of the invention, a least one such silent mutation results in expression of said cellulase at a higher level than a nucleotide sequence lacking at least one such mutation.

In another embodiment of the present invention, the invention comprises a nucleotide sequence from Thermotoga maritima having at least one mutation and having an increased expression level of a protein encoded by said nucleotide sequence compared to a Thermotoga maritima wild-type genomic sequence, wherein optionally, said mutation/s is silent.

In another embodiment of the present invention, the invention comprises a first nucleotide sequence encoding the polypeptide of SEQ ID NO:2 wherein said nucleotide sequence has been mutated with respect to a second sequence encoding SEQ ID NO:2 such that the expression level of said protein is increased relative to that of said protein encoded by said second nucleotide sequence.

A first nucleotide sequence encoding the polypeptide of SEQ ID NO:2 wherein said nucleotide sequence has been mutated with respect to a second sequence encoding SEQ ID NO:2 such that the expression level of said protein is increased relative to that of said protein encoded by said second nucleotide sequence.

In another embodiment of the present invention, the invention comprises a nucleotide sequence encoding a protein at least 50%, 60%, 70%, 80%, 90%, 95%, 97%, 98%, 99% or 100% identical to SEQ ID NO:2, or a fragment thereof, wherein said nucleotide sequence comprises at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, or any combination thereof.

In another embodiment of the present invention, any of the proteins of the invention are expressed in bacterial expression systems, wherein the bacteria expression system is a gram-negative bacteria expression system, e.g., Pseudomonas, E. coli, Ralstonia, or Caulobacter expression system.

In another embodiment of the present invention, expression of the cellulase of the invention is produced at least 1.0 g/L, 2.0 g/L, 3.0 g/L, 4.0 g/L, 5.0 g/L, 6.0 g/L, 7.0 g/L, 8.0 g/L, 9.0 g/L, 10.0 g/L, 11.0 g/L, 12.0 g/L, 13.0 g/L, 14.0 g/L, 15.0 g/L, 16.0 g/L, 17.0 g/L, 18.0 g/L, 19.0 g/L, 20.0 g/L, 21.0 g/L, 22.0 g/L, 23.0 g/L, 24.0 g/L, 25.0, g/L, 26.0 g/L, 27.0 g/L, 28.0 g/L, 29.0 g/L, 30.0 g/L, 31.0 g/L, 32.0 g/L, 33.0 g/L, 34.0 g/L, or 35.0 g/L.

In another embodiment of the present invention, a cellulase of the present invention is combined with a second enzyme wherein the second enzyme is selected from the group consisting of: a lactase, a lipase, a protease, a catalase, a xylanase, a cellulase, a glucanase, a mannanase, an amylase, an amidase, an epoxide hydrolase, an esterase, phospholipase, transaminase, an amine oxidase, cellobiohydrolase, an ammonia lyase, or any combination thereof.

In another embodiment of the present invention, the invention comprises an isolated, recombinant, or synthetic nucleotide, having a nucleic acid sequence comprising SEQ ID NO:1, wherein the nucleic acid sequence encodes a polypeptide having a cellulase activity.

In another embodiment of the present invention, the invention comprises an isolated, recombinant, or synthetic nucleotide, comprising a nucleic acid sequence of SEQ ID NO:1, wherein the nucleic acid sequence encodes a polypeptide having a cellulase activity and the polypeptide comprises an amino acid sequence of SEQ ID NO:2, or an enzymatically active fragment thereof.

In another embodiment of the present invention, the invention comprises, an isolated, recombinant, or synthetic nucleic acid sequence comprising SEQ ID NO:1 that encodes a polypeptide having a cellulase activity, wherein the polypeptide comprises an amino acid sequence of SEQ ID NO:2 and the polypeptide is produced in a recombinant Pseudomonas fluorescens expression system.

In another embodiment of the present invention, the present invention comprises a nucleotide sequence which encodes a polypeptide having cellulase activity, wherein the polypeptide is produced in a recombinant bacterial expression system.

In another embodiment of the present invention, the invention comprises a composition comprising a polypeptide encoded by SEQ ID NO:1, wherein optionally, the composition further comprises at least a second enzyme, and wherein optionally, the second enzyme is selected from the group consisting of: a lactase, a lipase, a protease, a catalase, a xylanase, a cellulase, a glucanase, a mannanase, an amylase, an amidase, an epoxide hydrolase, an esterase, phospholipase, transaminase, an amine oxidase, cellobiohydrolase, an ammonia lyase, or any combination thereof.

In another embodiment of the present invention, the invention comprises an isolated, recombinant, or synthetic nucleotide having a nucleic acid sequence comprising SEQ ID NO:1, wherein the nucleic acid sequence encodes a polypeptide having a cellulase activity.

In another embodiment of the present invention, the invention comprises an isolated, recombinant, or synthetic nucleotide comprising a nucleic acid sequence of SEQ ID NO:1, wherein the nucleic acid sequence encodes a polypeptide having a cellulase activity and the polypeptide comprises an amino acid sequence of SEQ ID NO:2, or an enzymatically active fragment thereof. In a further embodiment the isolated, recombinant, or synthetic nucleic acid sequence comprising SEQ ID NO:1 encodes a polypeptide having a cellulase activity, wherein the polypeptide comprises an amino acid sequence of SEQ ID NO:2 and the polypeptide is produced in a recombinant Pseudomonas fluorescens expression system.

In another embodiment of the present invention, the invention comprises, a composition comprising a polymeric viscosifier, a surfactant, a thermostabilizer, and an enzyme breaker comprising a wild-type cellulase derived from a hyperthermophilic bacterium or a mutated variant thereof. In further embodiment of the composition, the viscosifier is a guar gel comprising a linear guar, a crosslinked guar, or mixtures thereof. In further embodiment of the composition the enzyme breaker specifically hydrolyzes β-1,4 glycosidic bonds in the guar gel. In further embodiment of the composition the enzyme breaker does not specifically hydrolyze α-1,6 glycosidic bonds in the guar gel. In further embodiment of the composition the enzyme breaker retains its ability to hydrolyze β-1,4 glycosidic bonds in the guar gel at temperatures up to about 275° F. In further embodiment of the composition wherein the enzyme breaker retains its ability to hydrolyze β-1,4 glycosidic bonds in the guar gel at a pH of up to about 11. In further embodiment of the composition, the enzyme breaker has SEQ ID. NO. 2. In further embodiment of the composition, the enzyme breaker is encoded by a polynucleotide having SEQ ID. NO. 1. In further embodiment of any of the above compositions, the enzyme breaker is a mutated variant of the wild-type cellulase, and has a melting temperature that is at least 20° F. greater than the melting temperature of the wild type cellulase at about pH 6.5 and at least 10° F. greater than the melting temperature of the wild type cellulase at about pH 10.5. In a further embodiment, the enzyme breaker is a mutated variant comprising 12 mutations relative to the wild type cellulase. In a further embodiment, the variant cellulase comprises at least one of the following mutations F38Y, Y61Q, M69E, D70P, R71S, 194Q, I166V, S183R, S191A, E212P, L231V, M276A, E277S, R280G, T297P and T301Q.

In a further embodiment, said enzyme breaker is a mutant variant of a cellulase encoded by SEQ ID NO:3.

In a further embodiment, the invention comprises a method of reducing the viscosity of a polysaccharide gel comprising β-1,4 glycosidic bonds, the method comprising contacting the polysaccharide gel with a cellulase variant under permissive conditions and for a period of time sufficient to allow the cellulase to hydrolyze the β-1,4 glycosidic bonds in the polysaccharide gel thereby reducing the viscosity of the guar gel, wherein the cellulase variant comprises at least 12 mutations compared to a wild-type cellulase derived from a hyperthermophilic bacterium, and wherein the cellulase variant exhibits increased temperature and pH tolerance compared to the wild-type cellulase. In a further embodiment, said 12 mutations were generated by Gene Site Saturation Mutagenesis comprising repeated cycles of reductive reassortment, recombination, and selection. In an alternative embodiment, said polysaccharide gel comprises a linear guar, a crosslinked guar, or mixtures thereof. In an alternative embodiment said cellulase variant is encoded by a polynucleotide having SEQ ID. NO. 1. In an alternative embodiment, wherein permissive conditions comprise a temperature range of from about 180° F. to about 275° F.

In an alternative embodiment, wherein the permissive conditions comprise a pH range of from about 9 to about 11.

In a embodiment of the present invention, the invention comprises a method of treating a subterranean formation, comprising: introducing into the subterranean formation a fracturing fluid that comprises a polysaccharide gel and a cellulase variant; and allowing the gel and the cellulase variant to react under permissive conditions and for a period of time sufficient to allow the cellulase variant to hydrolyze at least some of the β-1,4 glycosidic bonds, but not the α-1,6 glycosidic bonds, in the gel, thereby generating a reduced viscosity fracturing fluid; wherein the cellulase variant comprises at least 12 mutations compared to a parental wild-type cellulase derived from a hyperthermophilic bacterium, and wherein the cellulase variant exhibits increased temperature and pH tolerance compared to the wild-type cellulase.

In a further embodiment of the above methods the hydrolysis with said cellulase variant comprises less residue that a comparable reduced viscosity fracturing fluid generated by a chemical breaker, wherein the dosage of cellulase variant and chemical breaker provide substantially the same reduction in viscosity.

In a further embodiment of the above methods wherein the reduced viscosity fracturing fluid generated by the cellulase variant retains greater conductivity than a comparable reduced viscosity fracturing fluid generated by a chemical breaker, wherein the dosage of cellulase variant and chemical breaker provide substantially the same reduction in viscosity.

### DESCRIPTION OF THE INVENTION

Enzymes are proteins that act as catalysts. Proteins are polymers of amino acids linked in dehydration reactions by peptide bonds. The identity of the amino acids and the order in which they are linked to form proteins determines a given protein's activity. This order in which amino acids are assembled into proteins (the protein “sequence”) is ultimately determined by the sequence of a DNA strand which “encodes” the protein.

The three-nucleotide sequence that specifies a given amino acid to be assembled into a protein is called a “codon.” The 20 amino acids built into proteins are collectively encoded by 64 tri-nucleotide codon sequences. The series of codons which specifies a protein is called an “Open Reading Frame.” An amino acid may be specified by as few as one or as many as six distinct codons. A change (or mutation) in the trinucleotide sequence of a codon that does not affect the amino acid specified is called a “silent” mutation.

As a result, there are many DNA sequences capable of encoding the same protein, because the DNA sequences differ from one another only through “silent” mutations. By altering one or more of the codons which encode a given protein, it may be possible to greatly increase the amount of protein which a gene produces without affecting the sequence of the protein that is encoded.

In some embodiments, the invention comprises SEQ ID NO:1. In some embodiments, the invention comprises the polynucleotide sequence of SEQ ID NO:1. In some embodiments, this sequence encodes a protein. In some embodiments, this protein is an enzyme having cellulase activity.

The improved nucleotide sequence disclosed herein is given as SEQ ID NO:1 and encodes a previously disclosed cellulase enzyme (SEQ ID NO:2) that was evolved from a parent cellulase enzyme isolated from a DNA library originating from Thermotoga maritima strain MSB8. The disclosed cellulase is described in PCT Publication No. WO 2009/020459, as SEQ ID NO:9 therein (encoded by the polynucleotide SEQ ID NO:8 therein, described herein as SEQ ID NO:3). In some embodiments, the invention comprises the polynucleotide sequence of SEQ ID NO:1, or fragments thereof. In some embodiments, these sequences encode a protein. In some embodiments, the protein is an enzyme having cellulase activity.

The invention comprises multiple nucleotide base changes with respect to SEQ ID NO:3. These changes are silent as to the encoded protein. The 14 base changes are set forth below. “Position” indicates the number of the nucleotide within the Open Reading Frame, with the first nucleotide of the first codon numbered as 1. In the event that the Open Reading Frame of SEQ ID NO:1 is joined to another nucleic acid sequence at its 5′ end so that the Open Reading Frame extends beyond the 5′ end of SEQ ID NO:1, the “Position” will continue to refer to the bases as numbered from the 5′ end of the Open Reading Frame of SEQ ID NO:1. Similarly, if the Open Reading Frame of SEQ ID NO:1 is truncated so that the Open Reading Frame does not begin at the 5′ end of a sequence related to SEQ ID NO:1, the numbering system will continue to originate from the 5′ end of said sequence corresponding to the 5′ end of SEQ ID NO:1.

The nucleotide base changes, or mutations, are specified using the notation (old nucleotide) (position) (new nucleotide). The mutations are as follows: T6C, T9C, T15G, A22C, G24T, A33C, A39C, A40C, A42C, A54C, A57C, T66C, G81A, A84C, or any combination thereof.

The base changes which distinguish SEQ ID NO:1 from prior reported sequences encoding the disclosed cellulase, collectively and individually, result in an Open Reading Frame which leads to a higher level of protein expression than previously employed nucleotide sequences encoding the same protein.

In some embodiments, a nucleotide sequence encoding a cellulase derived from Thermotoga maritima is disclosed, wherein the nucleotide sequence comprises at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, or any combination thereof. In some aspects of these embodiments, at least one mutation is silent as to the sequence of the encoded protein. In other aspects, at least one mutation results in the nucleotide sequence harboring at least one mutation directing expression of the cellulase at a higher level than a nucleotide sequence lacking the at least one mutation and not otherwise differing from the nucleotide sequence of the above.

In some embodiments, a nucleotide sequence encoding a cellulase is disclosed, wherein the nucleotide sequence comprises SEQ ID NO:3 and having at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, or any combination thereof.

In some embodiments, a nucleotide sequence from Thermotoga maritima is disclosed having at least one mutation which increases the expression level of a protein encoded by said nucleotide sequence compared to a Thermotoga maritima genomic sequence. In some aspects, at least one mutation is silent.

In some embodiments, a first nucleotide sequence encoding the polypeptide of SEQ ID NO:2 is disclosed wherein the nucleotide sequence has been mutated with respect to a second sequence encoding the polypeptide of SEQ ID NO:2 such that the expression level of the protein is increased relative to that of the protein encoded by the second nucleotide sequence.

Thermotoga maritima is a thermophilic eubacteria characterized by its ability to grow in extreme salt concentrations (i.e., from 0.25% NaCl to 6.00% NaCl). Thermotoga maritima belongs to the order Thermotogales whose members are thermophilic, rod-shaped, anaerobic and gram-negative. The minimum temperature for growth is around 55° C., optimum is 80°-85° C., and maximum is about 90° C. In some embodiments, the minimum temperature is less than 55° C. and the maximum temperature is greater than 90° C. These bacteria have slowly evolved from one of the deepest branches in the kingdom of eubacteria. Members of Thermotogales have been described as “wide-spread and cosmopolitan” (Huber, R. et al., 2006), thriving in active geothermal areas. Thermotoga maritima is closely related to the species Thermotoga neapolitana, Thermotoga peirophila, and Thermotoga napthophila. Specimens of Thermotoga maritima have been obtained from sea floors in Vulcano, Italy; Riberia Quente and Sao Miguel Island, Azores; Sangeang Island, Indonesia; and Fiji Island. (Huber, R. et al., 2006).

Strain MSB8 was isolated from a geothermally heated marine sediment at Vulcano, Italy (Huber, 1986). The temperature at the collection site ranged from 70-100° C., with a pH of 6.5-7.0. The strain has been deposited at the Deutsche Sammlung von Mikroorganismen as DSM 3109 and at ATCC as ATCC43589 (Huber, R. et al., 2006).

Thermotoga maritima strain MSB8 has been studied for its enzyme encoding genes due to the exceptional thermostability of the enzymes it produces. Liebl (Liebl, W. et al., 1996) has published an “Analysis of a Thermotoga maritima DNA fragment encoding two similar thermostable cellulases. CelA and CelB, and characterization of the recombinant enzymes.” Additionally, genes for amylolytic enzymes (Bibel, M. et al., 1998), reverse gyrase (Bouthier de la Tour, C. et al., 1998), alpha-amylase (Liebl, W. et al., 1997), alpha-glucuronidase (Ruile, P. et al., 1997), xylanase (Winterhalter. C. et al., 1995), beta-glucosidase (Liebl, W, et a., 1994), glucanotransferase (Liebl, W. et al., 1992) have been isolated and analyzed. A study by Bronnenmeier (Bronnenmeier, K. et al., 1995), “Purification of Thermotoga maritima enzymes for the degradation of cellulosic materials” has shown that these enzymes are of value for degrading cellulose and xylan.

### Expression Systems

In some embodiments, the DNA encoding the cellulase of the present invention may be introduced, either on a plasmid or stably transformed into the genome of, for example, any number of gram negative bacterial systems such as E. coli, Pseudomonas species such as fluorescens, Pseudomonas putida, Pseudomonas aeruginosa, Ralstonia species, or Caulobacter species. Similarly, the cellulase may be introduced into any number of gram positive bacterial expression systems such as Bacillus species such as Bacillus subtilis, Bacillus megaterium, Bacillus brevis, Lactococcus species such as Lactococcus lactis, Lactobacillus species, Streptomyces species such as Streptomyces lividans. Other gram negative, gram positive or unrelated eubacterial or archaeal expression systems may be used to express the cellulase.

In some embodiments, SEQ ID NO:1 is used to direct an increased level of expression in a number of systems in which the disclosed cellulase protein may be expressed. SEQ ID NO:1 may be introduced into any number of expression systems to express the disclosed cellulase at an improved accumulation level. For example, SEQ ID NO:1 may be introduced, either on a plasmid or stably transformed into the genome of, for example, any number of gram negative bacterial systems such as E. coli, Pseudomonas species such as fluorescens, Pseudomonas putida, Pseudomonas aeruginosa, Ralstonia species, or Caulobacter species. Similarly, SEQ ID NO:1 may be introduced into any number of gram positive bacterial expression systems such as Bacillus species such as Bacillus subtilis, Bacillus megaterium, Bacillus brevis. Lactococcus species such as lactococcus lactis, lactobacillus species, Streptomyces species such as Streptomyces lividans. Other gram negative, gram positive or unrelated eubacterial or archaeal expression systems may be used to express SEQ ID NO:1. In a further embodiment, SEQ ID NO:1 may be introduced into any number of eukaryotic expression systems such as Saccharomyces, Schizosaccharomyces pombe, Pichia pastoris, and Hansanuela polymorpha.

More specifically. SEQ ID NO:1 may be introduced into a plasmid to direct its expression. Plasmids to which SEQ ID NO:1 may be introduced include, for example, E. coli expression vectors of the families pQE, pET, and pASK; Pseudomonas expression vectors of the families pCN51 LT8, RSF1010, pWZ112T, and pMYC; Bacillus expression vectors of the families pBAX, pHT01, and pHIS1525; Streptomyces expression vectors of the families pIJ6021 and pIJ2460; and Lactococcus: expression vectors of the families pNZ9530 and pNZ8148, for example. These examples are for demonstrative purposes and do not represent a complete set of vectors in which the polynucleotide sequence of SEQ ID NO:1 can be expressed.

In some embodiments, the expression system could be any Pseudomonas fluorescens expression system known in the art, for example, the Pseudomonas fluorescens expression system that is commercially available from Dow Global Technologies Inc., strain DC454 (US Patent PUB. APP. NO. 20050130160 and US Patent PUB. APP. NO. 20050186666). A nucleic acid sequence encoding the cellulase enzyme or polypeptide is inserted either in the pMYC vector (Dow Global Technologies Inc., US Patent PUB. APP. NO. 20050130160) or in the pDOW1169 vector (Dow Global Technologies Inc., US Patent PUB. APP. NO. 20080058262) and then introduced into the Pseudomonas fluorescens host by electroporation. Those skilled in the art will know alternative vectors that can be used as embodiments of this invention.

In some embodiments, the cellulase will be expressed at least at the following expression levels: 1.0 g/L, 2.0 g/L, 3.0 g/L, 4.0 g/L, 5.0 g/L, 6.0 g/L, 7.0 g/L, 8.0 g/L, 9.0 g/L, 10.0 g/L, 11.0 g/L, 12.0 g/L, 13.0 g/L, 14.0 g/L, 15.0 g/L, 16.0 g/L, 17.0 g/L, 18.0 g/L, 19.0 g/L, 20.0 g/L, 21.0 g/L, 22.0 g/L, 23.0 g/L, 24.0 g/L, 25.0, g/L, 26.0 g/L, 27.0 g/L, 28.0 g/L, 29.0 g/L, 30.0 g/L, 31.0 g/L, 32.0 g/L, 33.0 g/L, 34.0 g/L, 35.0 g/L. or more.

### Nucleic Acid

The invention provides isolated, synthetic, or recombinant nucleic acids comprising sequences completely complementary to the nucleic acid sequences of the invention (complementary (non-coding) and coding sequences also hereinafter collectively referred to as nucleic acid sequences of the invention).

The invention provides isolated, synthetic, or recombinant nucleic acids comprising a nucleic acid encoding at least one polypeptide having a cellulolytic activity, wherein the nucleic acid comprises a sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity (homology) to an exemplary nucleic acid of the invention, including the sequence of SEQ ID NO:1. For example, the invention provides isolated, synthetic, or recombinant nucleic acids comprising a nucleic acid sequence SEQ ID NO:1 (the exemplary polynucleotide sequence of this invention). The invention provides isolated, synthetic, or recombinant nucleic acids encoding a polypeptide comprising a sequences as set forth in SEQ ID NO:2 (the exemplary polypeptide sequences of this invention), and enzymatically active fragments thereof.

### Polypeptide

Polypeptides and peptides of the invention are isolated, synthetic, or recombinant polypeptides. Peptides and proteins can be recombinantly expressed in vitro or in vivo. The peptides and polypeptides of the invention can be made and isolated using any method known in the art. Polypeptides and peptides of the invention can also be synthesized, whole or in part, using methods well known in the art. For example, cellulase polypeptides can be produced in a standard recombinant expression system (as described herein), chemically synthesized, or purified from organisms in which they are naturally expressed.

The invention provides isolated, synthetic, or recombinant polypeptides having cellulolytic activity comprising an amino acid sequence having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or has 100% (complete) sequence identity to an exemplary amino acid sequence of the invention (e.g., SEQ ID NO:2), or an enzymatically active fragment thereof.

The invention provides isolated, synthetic, or recombinant polypeptides comprising a sequence as set forth in SEQ ID NO:2, and enzymatically active fragments thereof, and variants thereof.

In alternative embodiments, the invention provides polypeptides (and the nucleic acids that encode them) having cellulolytic activity but lacking a signal sequence, a prepro domain, a dockerin domain, and/or a carbohydrate binding module (CBM); and in one aspect, the carbohydrate binding module (CBM) comprises, or consists of, a cellulose binding module, a lignin binding module, a xylan binding module, a xylose binding module, a mannose binding module, a xyloglucan-specific module, and/or a arabinofuranoside binding module.

In alternative embodiments, the invention provides polypeptides (and the nucleic acids that encode them) having a cellulolytic activity further comprising a heterologous sequence; and in one aspect, the heterologous sequence comprises, or consists of a sequence encoding: (i) a heterologous signal sequence, a heterologous carbohydrate binding module, a heterologous dockerin domain, a heterologous catalytic domain (CD), or a combination thereof; (ii) the sequence of (i), wherein the heterologous signal sequence, carbohydrate binding module or catalytic domain (CD) is derived from a heterologous enzyme; or, (iii) a tag, an epitope, a targeting peptide, a cleavable sequence, a detectable moiety or an enzyme; and in one aspect, the heterologous carbohydrate binding module (CBM) comprises, or consists of, cellulose binding module, a lignin binding module, a xylan binding module, a xylose binding module, a mannose binding module, a xyloglucan-specific module and/or a arabinofuranoside binding module; and in one aspect, the heterologous signal sequence targets the encoded protein to a vacuole, the endoplasmic reticulum, a chloroplast or a starch granule.

### Enzymatic Activity

The enzymatic hydrolysis of pNP-β-D-lactopyranoside by the disclosed cellulase can be used as a measure of activity of the enzyme. The liberation of p-nitrophenol can be followed spectrophotometrically at 405 nm. The increase in absorbance at 405 nm can be converted to μmoles of p-nitrophenol by using a standard absorbance at those defined conditions. One unit of activity is defined as the quantity of enzyme required to liberate 0.42 μmole of p-nitrophenol from 2 mM pNP-β-D-lactopyranoside during one minute at pH 7.00 and 80° C. (Advances in Carbohydrate Chemistry and Biochemistry, Academic Press, 1999)

### Thermostability

In some aspects, the recombinant nucleic acid of the present invention encodes a polypeptide having a cellulolytic activity that is thermostable. For example, a polypeptide of the invention. SEQ ID NO:2, or the variant evolved enzymes of the invention can be thermostable. The thermostable polypeptide according to the invention can retain binding and/or enzymatic activity, e.g., cellulolytic activity, a under conditions comprising a temperature in the range from greater than 37° C. to about 95° C. or between about 55° C. to about 85° C., or between about 70° C. to about 75° C., or between about 70° C. to about 95° C., between about 90° C. to about 95° C., between about 95° C. to about 105° C., or between about 95° C. to about 110° C. In some aspects, wherein the polypeptide can retain binding and/or enzymatic activity, e.g., cellulolytic activity, under conditions comprising 1° C. to about 5° C., between about 5° C. to about 15° C., between about 15° C. to about 25° C., between about 25° C. to about 37° C. In some aspects polypeptides of the invention can retain binding and/or enzymatic activity, e.g., cellulolytic activity, under conditions comprising 90° C., 91° C., 92° C., 93° C., 94° C., 95° C., 96° C., 97° C., 98° C., 99° C., 100° C., 101° C., 102° C., 103° C., 103.5° C. 104° C., 105° C., 107° C., 108° C., 109° C. or 110° C., or more. In some embodiments, the thermostable polypeptides according to the invention retains activity, e.g., a cellulolytic activity at a temperature in the ranges described above, under acidic conditions comprising about pH 6.5, pH 6, pH 5.5, pH 5, pH 4.5 or pH 4 or less (more acidic), or, retain a cellulolytic activity after exposure to acidic conditions comprising about pH 6.5, pH 6, pH 5.5, pH 5, pH 4.5 or pH 4 or less (more acidic); or, retain activity under basic conditions comprising about pH 7, pH 7.5 pH 8.0, pH 8.5, pH 9, pH 9.5, pH 10, pH 10.5, pH 11, pH 11.5, pH 12, pH 12.5 or more (more basic) or, retain a cellulolytic activity after exposure to basic conditions comprising about pH 7, pH 7.5 pH 8.0, pH 8.5, pH 9, pH 9.5, pH 10, pH 10.5, pH 11, pH 11.5, pH 12, pH 12.5 or more (more basic).

### Thermotolerance

In some aspects, the recombinant nucleic acid of the present invention encodes a polypeptide having a cellulolytic activity that is thermotolerant. For example, a polypeptide of the invention. SEQ ID NO:2, or the variant evolved enzymes of the invention can be thermotolerant. In some aspects, the cellulolytic activity is thermotolerant. e.g., wherein the polypeptide retains cellulolytic activity after exposure to a temperature in the range from greater than 37° C. to about 95° C., or between about 55° C. to about 85° C., or between about 70° C. to about 75° C., or between about 70° C. to about 95° C., between about 90° ° C. to about 95° C., between about 95° C. to about 105° C., or between about 95° C. to about 110° C. In some aspects, wherein the polypeptide retain a cellulolytic activity after exposure to conditions comprising a temperature range of between about ° PC to about 5° C., between about 5° C. to about 15° C., between about 15° C. to about 25° C., between about 25° C. to about 37° C. In some aspects polypeptides of the invention can retain a cellulolytic activity after exposure to a temperature up to 90° C., 91° C., 92° C., 93° C., 94° C., 95° C., 96° C., 97° C., 98° C., 99° C. 100° C., 101° C., 102° C., 103° C., 104° C., 105° C., 107° C., 108° C., 109° C., or 110° C., or more. In some aspects, the polypeptides encoded by nucleic acids of the invention retain cellulolytic activity under acidic conditions comprising about pH 6.5, pH 6, pH 5.5, pH 5, pH 4.5 or pH 4 or less (more acidic), or, retain a cellulolytic activity after exposure to acidic conditions comprising about pH 6.5, pH 6, pH 5.5, pH 5, pH 4.5 or pH 4 or less (more acidic); or, retain a cellulolytic activity under basic conditions comprising about pH 7, pH 7.5 pH 8.0, pH 8.5, pH 9, pH 9.5, pH 10, pH 10.5, pH 11, pH 11.5, pH 12, pH 12.5 or more (more basic).

### Cellulosic Digestion

In some aspects, the compositions and methods of the invention are used in the enzymatic digestion of biomass and can comprise use of many different enzymes, including the cellulases and hemicellulases. Cellulases used to practice the invention can digest cellulose to glucose. In some aspects, compositions used to practice the invention can include mixtures of enzymes, e.g., xylanases, xylosidases (e.g., β-xylosidases), cellobiohydrolases, and/or arabinofuranosidases, or other enzymes that can digest hemicelluloses, cellulose, and lignocellulosic material, to fermentable sugars and/or to monomer sugars.

Enzymes, e.g., endoglucanases, of the invention are used to digest cellulose or any beta-1,4-linked glucan-comprising synthetic or natural material, including those found in any plant material. Enzymes, e.g., endoglucanases, of the invention are used as commercial enzymes to digest cellulose from any source, including all biological sources, such as plant biomasses, e.g., corn, grains, grasses (e.g., Indian grass, such as Sorghastum nutans, or, switch grass. e.g., Panicum species, such as Panicum virgatum), or, woods or wood processing byproducts, e.g., in the wood processing, pulp and/or paper industry, in textile manufacture and in household and industrial cleaning agents, and/or in biomass waste processing.

### Dietary

In some embodiments, the cellulase of the present invention may be used to pre-treat, modify, digest, or enhance the digestion of, a food, food additive, or dietary supplement for animals or human beings. In some embodiments, the cellulase of the present invention may be used as a food, food additive, or dietary supplement for animals or human beings. In some aspects the cellulase will treat or will act as a prophylaxis for digestive disorders. In another aspect of the present invention the cellulase will alter or enhance digestion. In another aspect of the present invention the cellulase will enhance, alter, or aid in the digestion of foodstuffs. In a further aspect of the invention the cellulase will enhance, aid, or alter the nutrient value of foodstuffs. In a further aspect, the cellulase is active in the digestive tract, e.g., in a stomach and/or intestine.

In some embodiments, the cellulase of the invention may be used as an animal feed or an animal feed additive. In some embodiments the thermostability and or thermotolerance of the cellulase allows for the formation of pellets without the need for a secondary agent such as salt or wax. An animal feed comprising a cellulase can be provided to an animal in any formulation known to those skilled in the art. Examples of animal feed formulations include, but are not limited to: a delivery matrix, a pellet, a tablet, a gel, a liquid, a spray, ground grain, or a powder.

The invention provides edible enzyme delivery matrix comprising a thermostable recombinant cellulase enzyme, e.g., a polypeptide of the invention. The invention provides methods for delivering a cellulase supplement to an animal, the method comprising: preparing an edible enzyme delivery matrix in the form of pellets comprising a granulated edible carrier and a thermostable recombinant cellulase enzyme, wherein the pellets readily disperse the cellulase enzyme contained therein into aqueous media, and administering the edible enzyme delivery matrix to the animal. The recombinant cellulase enzyme can comprise a polypeptide of the invention. The granulate edible carrier can comprise a carrier selected from the group consisting of a grain germ, a grain germ that is spent of oil, a hay, an alfalfa, a timothy, a soy hull, a sunflower seed meal and a wheat midd. The edible carrier can comprise grain germ that is spent of oil. The cellulase enzyme can be glycosylated to provide thermostability at pelletizing conditions. The delivery matrix can be formed by pelletizing a mixture comprising a grain germ and a cellulase. The pelletizing conditions can include application of steam. The pelletizing conditions can comprise application of a temperature in excess of about 80° C. for about 5 minutes and the enzyme retains a specific activity of at least 350 to about 900 units per milligram of enzyme.

### Methods of Making Ethanol

The invention provides methods for making ethanol comprising contacting a starch—comprising composition with a polypeptide having a cellulolytic activity, wherein the polypeptide has a sequence of the invention, or the polypeptide is encoded by a nucleic acid comprising a sequence of the invention, or an enzymatically active fragment thereof. The invention provides compositions comprising a starch and a polypeptide having a cellulolytic activity, wherein the polypeptide has a sequence of the invention, or the polypeptide is encoded by a nucleic acid comprising a sequence of the invention, or an enzymatically active fragment thereof.

### Brewing and Fermenting

The invention provides methods of brewing (e.g., fermenting) beer comprising the cellulase of the invention. In one exemplary process, starch-containing raw materials are disintegrated and processed to form a malt. An enzyme of the invention is used at any point in the fermentation process. The cellulase of the invention can be used in the brewing industry for the degradation of beta-glucans. In some aspects, the cellulases of the invention are used in the brewing industry for the clarification of the beverage. Enzymes of the invention can be used in the beverage industry in improving filterability of wort or beer, as described, e.g., in U.S. Pat. No. 4,746,517.

In some aspects, the cellulase of the invention is used in mashing and conversion processes. In the brewing and fermentation industries, mashing and conversion processes are performed at temperatures that are too low to promote adequate degradation of water-soluble glucans, mannans, arabinoxylans or xylans, or other polysaccharides. These polymers form gummy substrates that can cause increased viscosity in the mashing wort, resulting in longer mash run-off, residual haze and precipitates in the final beer product due to inefficient filtration and low extraction yield.

In some aspects, the cellulase of the invention are used in malthouse operations, e.g., glucanase is added to the process water, to shorten germination times and/or to encourage conversion of poor quality barley to acceptable malts. In some aspects, enzymes of the invention are used for mashing, e.g., they are added to increase wort filterability and/or improve lautering (separating the won from the mash). In some aspects, enzymes of the invention are used in the fermentor and/or settling tank to, e.g., assist in haze clearing and/or to improve filtration. In some aspects, enzymes of the invention are used in adjunct brewing, e.g., a glucanase of the invention is added to breakdown glucans, mannans, arabinoxylans, or xylans, or other polysaccharides from barley, wheat, and/or other cereals, including glycans in malt. In some aspects, enzymes of the invention are used in malt brewing, e.g., a glucanase of the invention is added to modify poor malts with high glucan content.

The cellulase of the invention can be used in any beer or alcoholic beverage producing process, as described, e.g., in U.S. Pat. Nos. 5,762,991; 5,536,650; 5,405,624; 5,021,246; 4,788,066.

### Treating Foods and Food Processing

The cellulases of the invention have numerous applications in food processing industry. For example, in one aspect, the enzymes of the invention are used to improve the extraction of oil from oil-rich plant material, e.g., oil-rich seeds, for example, soybean oil from soybeans, olive oil from olives, rapeseed oil from rapeseed and/or sunflower oil from sunflower seeds.

The cellulase of the invention can be used for separation of components of plant cell materials. For example, enzymes of the invention can be used in the separation of glucan-rich material (e.g., plant cells) into components. In some aspects, enzymes of the invention can be used to separate glucan-rich or oil-rich crops into valuable protein and oil and hull fractions. The separation process may be performed by use of methods known in the art.

The cellulase of the invention can be used in the preparation of fruit or vegetable juices, syrups, extracts and the like to increase yield. The enzymes of the invention can be used in the enzymatic treatment (e.g., hydrolysis of glucan—comprising plant materials) of various plant cell wall-derived materials or waste materials, e.g. from cereals, grains, wine or juice production, or agricultural residues such as vegetable hulls, bean hulls, sugar beet pulp, olive pulp, potato pulp, and the like. The enzymes of the invention can be used to modify the consistency and appearance of processed fruit or vegetables. The enzymes of the invention can be used to treat plant material to facilitate processing of plant material, including foods, facilitate purification or extraction of plant components. The cellulase of the invention can be used to improve feed value, decrease the water binding capacity, improve the degradability in waste water plants and/or improve the conversion of plant material to ensilage, and the like. The cellulase of the invention can also be used in the fruit and brewing industry for equipment cleaning and maintenance.

### Detergent Compositions

The invention provides detergent compositions comprising one or more polypeptides of the invention and methods of making and using these compositions. The invention incorporates all methods of making and using detergent compositions, see, e.g., U.S. Pat. Nos. 6,413,928; 6,399,561; 6,365,561; 6,380,147. The detergent compositions can be a one and two part aqueous composition, a non-aqueous liquid composition, a cast solid, a granular form, a particulate form, a compressed tablet, a gel and/or a paste and a slurry form. The invention also provides methods capable of a rapid removal of gross food soils, films of food residue and other minor food compositions using these detergent compositions. Enzymes of the invention can facilitate the removal of starchy stains by means of catalytic hydrolysis of the starch polysaccharide. Enzymes of the invention can be used in dishwashing detergents in textile laundering detergents. The actual active enzyme content depends upon the method of manufacture of a detergent composition and is not critical, assuming the detergent solution has the desired enzymatic activity. In some aspects, the amount of glucosidase present in the final solution ranges from about 0.001 mg to 0.5 mg per gram of the detergent composition. The particular enzyme chosen for use in the process and products of this invention depends upon the conditions of final utility, including the physical product form, use pH, use temperature, and soil types to be degraded or altered. The enzyme can be chosen to provide optimum activity and stability for any given set of utility conditions. The detergents of the invention can comprise cationic, semi-polar nonionic, or zwitterionic surfactants; or, mixtures thereof.

The present invention provides cleaning compositions including detergent compositions for cleaning hard surfaces, detergent compositions for cleaning fabrics, dishwashing compositions, oral cleaning compositions, denture cleaning compositions, and contact lens cleaning solutions. In some aspects, the invention provides a method for washing an object comprising contacting the object with a polypeptide of the invention under conditions sufficient for washing. A polypeptide of the invention may be included as a detergent additive. The detergent composition of the invention may, for example, be formulated as a hand or machine laundry detergent composition comprising a polypeptide of the invention. A laundry additive suitable for pre-treatment of stained fabrics can comprise a polypeptide of the invention. A fabric softener composition can comprise a polypeptide of the invention. Alternatively, a polypeptide of the invention can be formulated as a detergent composition for use in general household hard surface cleaning operations.

### Oil and Gas Exploration and Clean-Up

To increase the productivity of oil and gas wells and shale gas reservoirs, a highly specialized technique called “hydraulic fracturing” is being increasingly utilized. In a typical hydraulic fracturing operation, large volumes of guar-based fluid (in gel form and referred to as “fracturing fluid”) are pumped into the wellbore under very high hydrostatic pressure. The pressurized fluid creates new fissures and fractures in the formation surrounding the wellbore. The sand particles contained in the fracturing fluid move and settle into the newly-created fractures and function to prop these channels open thus increasing oil and gas flow. Once the sand is deposited into the fractures, the gel has to be degraded (i.e., broken down) and brought back up to the surface so as to remove any blockage to the flow of oil or gas. Industry uses viscosity breakers (such as oxidizers, acids, or enzymes) to degrade the fracturing fluid and to remove any solid gel residue from the fissures and fractures.

In some embodiments, the disclosed cellulase will be used as a high temperature viscosity breaker to enhance oil and gas operations. More specifically, the disclosed cellulase of the present invention will be applied to a fracturing fluid when hydraulic fracturing is performed in oil or gas wells.

The enzyme encoded by SEQ ID NO:1, as well as cellulases encoded by other polynucleotides disclosed herein, or obtainable by methods disclosed herein, may potentially be used to hydrolyze a broad spectrum of polysaccharides—many of which are useful in oil and gas drilling, fracturing and well clean-up operations. The disclosed cellulases exhibit broad spectrum β-glycosidase activity. e.g., against guar, hydroxypropyl guar, carboxymethyl guar, carboxymethyl hydroxypropyl guar, carboxymethyl cellulose, barley β-glucan, and locust bean gum. The enzyme activity pattern is preferably both endo and exo, allowing effective reduction in the viscosity of polysaccharides. e.g., guar and derivatized guar solutions, by cleaving within long polysaccharide chains and also by cleaving disaccharide units from the ends of the polymers. Besides the aforementioned polysaccharides, other substrates of the disclosed enzymes include those capable of forming linear or cross-linked gels. Examples of suitable polysaccharide substrates include glactomnannan gums, guars, derivatized guars, cellulose and cellulose derivatives, starch, starch derivatives, xanthan, derivatized xanthan and mixtures thereof. Specific examples also include, but are not limited to, guar gum, guar gum derivative, locust bean gum, karaya gum, xanthan gum, cellulose and cellulose derivatives, etc. Typical polymers or gelling agents to which the disclosed enzymes may be directed include guar gum, hydroxypropyl guar, carboxymethyl hydroxypropyl guar, hydroxyethyl cellulose, carboxymethyl hydroxyethyl cellulose, carboxymethyl cellulose, dialkyl carboxymethyl cellulose, etc. Other examples of polymers include, but are not limited to, phosphomannans, scleroglucans, dextrans and other types of polymers. In some embodiments, a polymer substrate is carboxymethyl hydroxypropyl guar. In some embodiments, a disclosed enzyme may also be effective in hydrolyzing biogums (e.g., succinoglycan biogums made from date syrup or sucrose). In some embodiments, a disclosed enzyme may be used to hydrolyze cellulose-containing or derivatized cellulose-containing polymers—typically, the enzymes attack glucosidic linkages of the cellulose backbone. The disclosed enzymes may be suitable for degrading the polymer into mostly monosaccharide units, in some cases, by specifically hydrolyzing the exo(1,4)-β-D-glucosidic and endo(1,4)-β-D-glucosidic linkages between monosaccharide units and the cellulose backbone in the (1,4)-β-D-glucosidic linkages of any cellobiose fragments.

In each fracturing job that uses the disclosed cellulases, field operators will generally first perform an enzyme dose optimization study in an industrial lab. Such studies may include dilution of the cellulase to a concentration of 10˜400 ppm and mixed with linear or cross-linked guar gum (25-60 lb/1,000 gal). Depending on the application conditions, guar gum maybe cross-linked using a cross-linker, especially for wells where higher temperature, pressure, and pH conditions are present. The enzyme dose information resulting from such optimization studies is then used in the actual fracturing job.

The unique activity of the disclosed cellulase allows for the hydrolysis of guar-based fracturing fluids in a smooth and controlled manner in deep wells, where high temperature and high pH conditions are present. Compared to chemical breakers, the disclosed cellulase of the present invention provides a non-corrosive and environmentally benign alternative to the harsh and non-selective chemical breakers.

In some embodiments of the present invention the cellulase may be used to treat, clean, or alter fluids used in oil and gas exploration activities. In a further aspect the cellulase of this invention will treat or alter the fluids, in part, or completely, so that the fluids may be used again, or recycled, for use in additional oil and gas exploration activities or to be disposed of in an environmentally friendly way.

Described herein are compositions for uses including oil and gas fracturing procedures. In some embodiments, the compositions include one or more cellulase enzymes derived from hyperthermophilic bacteria and/or non-naturally occurring variants thereof, in combination with a polymeric viscosifier. In some embodiments, the compositions may optionally include additional ingredients including agents to control fluid loss, reduce formation damage, adjust pH, control microbial growth and improve temperature stability. Some typical additional ingredients include acids, anti-bacterial agents, clay stabilizer, corrosion inhibitor, crosslinker, friction reducer, iron control, scale inhibitor, surfactants and thermal stabilizers (e.g., sodium thiosulfate).

In some embodiments, the cellulase enzymes described herein possess glucanase, e.g., endoglucanase, mannanase, xylanase activity or a combination of these activities. In some aspects, the glucanase activity is an endoglucanase activity (e.g., endo-1,4-beta-D-glucan 4-glucano hydrolase activity) and comprises hydrolysis of 1,4-beta-D-glycosidic linkages in cellulose, cellulose derivatives (e.g., carboxy methyl cellulose and hydroxy ethyl cellulose) lichenin, beta-1,4 bonds in mixed beta-1,3 glucans, such as cereal beta-D-glucans or xyloglucans and other plant material containing cellulosic parts. In alternative aspects, these glucanases e.g., endoglucanases, mannanases, xylanases have increased activity and stability, including thermotolerance or thermostability, at increased or decreased pHs and temperatures.

In some embodiments, the enzyme breakers described may be encapsulated to stabilize the enzyme, improve thermostability and alkaline pH tolerance, and provide controlled release. An encapsulated breaker having a coating or membrane that hydrolytically degrades may be superior to prior art enzyme breaker systems, because it could allow better control of release time and ease of handling not previously afforded. For example, because the breaker is encapsulated in a material that reacts with water, rather than simply dissolves or dissipates in water, the release can be controlled through the reaction rate of the coating with water. Likewise, by insulating the enzyme from the harsh down-hole conditions (high temperature and pH) for some period of time, can provide delayed and complete viscosity breaks. Those skilled in the art will appreciate that the reaction rate of the coating (and therefore the breaker release profile) can be varied broadly depending on the encapsulating polymer chemistry employed. Examples of breaker encapsulation compositions and methods are provided in U.S. Pat. Nos. 5,164,099, 6,163,766, 5,373,901, 5,437,331, and 6,357,527, the disclosures of each of which are incorporated herein by reference thereto.

### DNA Sequences and Encoded Cellulase Sequences

Some cellulases derived from hyperthermophilic bacteria and/or non-naturally occurring variants thereof are described in PC publication WO 2009/02049; the entire disclosure of which is incorporated herein by reference thereto. Included within the entire specification of the WO 2009/02049 publication, the entirety of which is hereby incorporated by reference are the below-listed DNA and amino acid SEQ ID NOS. These include:
WO 2009/02049 SEQ ID NOS: 1, 2 (wild-type ‘parent’ T. maritima cellulase), disclosed herein as SEQ ID NOs:5 and 6.
WO 2009/02049 SEQ ID NOS: 3 (wild-type DNA, altered to remove alternate starts) disclosed herein as SEQ ID NO:7.
WO 2009/02049 SEQ ID NOS: 6, 7 (“7X” combined Gene Site Saturation Mutagenesis (“GSSM”) mutations) disclosed herein as SEQ ID NOs:8 and 9.
WO 2009/02049 SEQ ID NOS: 8, 9 (“12X-6” combined GSSM mutations), disclosed herein as SEQ ID NOs:3 and 2.
WO 2009/02049 SEQ ID NOS: 10, 11 (“13X-1” combined GSSM mutations) disclosed herein as SEQ ID NOs:10 and 11.
WO 2009/02049 SEQ ID NOS: 12, 13 (“12X-1” combined GSSM mutations) disclosed herein as SEQ ID NOs:12 and 13.
WO 2009/02049 SEQ ID NOS: 16, 17 (alternative cellulase breaker from Thermotoga sp.) disclosed herein as SEQ ID NOs:14 and 15.
WO 2009/02049 SEQ ID NOS: 18, 19 (“7X” codon-optimized version of T. maritima cellulase for maize expression) disclosed herein as SEQ ID NOs:16 and 17.
WO 2009/02049 SEQ ID NOS: 20, 21 (“12X-6” codon-optimized version of T. maritima cellulase for maize expression) disclosed herein as SEQ ID NOs:18 and 19.

Besides the above-listed nucleotide and amino acid sequences related to wild-type and evolved variants of the cellulase from Thermotoga maritima strain MSB8, the additional mutants listed in Table 2 and Example 5 (from WO 2009/02049, and excerpted or reproduced below) are also deemed useful as components of the compositions described herein and/or in the methods of making these compositions.

Enzyme Activity Assays (Example 5 from WO 2009/02049)

The following example describes exemplary enzymes, variants of the “parental” or “wild type” protein identified in WO 2009/02049 as SEQ ID NO:2, and data demonstrating their activity; the Figures and data are incorporated by reference.

Sequences are provided having specific residue changes to the “parent” (or “wild type”) SEQ ID NO:6 (corresponding to WO 2009/02049 SEQ ID NO:2) encoded, e.g., by SEQ ID NO:5 (corresponding to WO 2009/02049 SEQ ID NO:1), as summarized (in part) in Table 1, above, and Tables 2 and 3, below.

TABLE 2

| | Position: | | | | | | | | | | | | | | | |
|---|---|---|---|---|---|---|---|---|---|---|---|---|---|---|---|---|
| | 38 | 61 | 69 | 70 | 71 | 94 | 166 | 183 | 191 | 212 | 231 | 276 | 277 | 280 | 297 | 301 |
| Mutation: | Y | Q | E | P | S | Q | V | R | A | P | V | A | S | G | P | Q |
| 7X | Y | Q | E | | | Q | | R | A | | | A | | | | |
| 10X-1 | Y | Q | E | | | Q | V | R | A | P | | A | | G | | |
| 10X-2 | Y | Q | E | | | Q | V | | A | P | | A | | G | P | |
| 11X-1 | Y | Q | E | | | Q | V | R | A | P | | A | | G | P | |
| 11X-2 | | Q | | | | Q | V | R | A | P | | A | | G | P | Q |
| 12X-1 | | Q | | | S | Q | V | R | A | P | | A | | G | P | Q |
| 12X-2 | Y | Q | | | S | Q | | R | A | P | | A | | G | P | Q |
| 12X-3 | | Q | | P | S | Q | V | R | A | P | | A | | G | P | Q |
| 12X-4 | Y | Q | | | S | Q | V | R | A | P | | A | | G | P | Q |
| 12X-5 | | Q | E | P | | | V | R | A | P | | A | | G | P | Q |
| 12X-6 | | Q | E | P | S | | V | R | A | P | | A | | G | P | Q |
| 12X-7 | | Q | E | | | Q | V | R | A | P | V | A | | G | P | Q |
| 13X-1 | Y | Q | E | | S | | V | R | A | P | V | A | | G | P | Q |
| 13X-2 | Y | Q | E | P | S | Q | V | | A | P | | A | | G | P | Q |
| 13X-3 | Y | Q | | P | S | Q | V | R | A | P | | A | | G | P | Q |
| 13X-4 | Y | Q | E | P | | Q | V | R | A | P | V | A | | G | P | |
| 13X-5 | Y | Q | E | P | S | Q | V | R | A | | | A | | G | P | Q |
| 13X-6 | Y | Q | E | P | | Q | V | R | A | P | | A | S | G | P | |
| 13X-7 | Y | Q | E | P | S | Q | | R | A | P | | A | | G | P | Q |
| 14X | Y | Q | E | P | S | Q | V | R | A | P | V | A | | G | P | |

```
TABLE 3
MGVDPFERNKILGRGINIGNALEAPNEGDWGVVIKDEFFDIKEAGFSHVRP
                                                                            ↓
                                                                            Y
IRWSTHAYAFPPYKIMDRFFKRVDEVINGALKRGLAVVNHHYEELMNDPEE
              ↓  ↓↓↓  ↓
              Q EPS Q
HKERFLALWKQIADRYKDYPETLFFEILNEPHGNLTPKWNELLEEALKVRS
IDKKHT [symbol] GTAEWGGISALEKLSVPKWEKNSIVTHYYNPFEFTHQGAEWVE
            ↓  ↓  ↓  ↓
            V R A P
GSEKWLGRKWGSPDDQKHLEEFNFEEWSKKNKRPIYIGEFGAYRKADLESR
          ↓
          V
KWTSFVVREMEKRRWSWAYWEFCSGFGVYDTLRKTWNKDLLEALIGGDSIE
                  ↓↓  ↓  ↓  ↓
                  AS G P Q [symbol]
indicates data missing or illegible when filed
```

Thermal tolerance of variants was measured using purified enzyme compared to the parental “wild-type” SEQ ID NO:6 (WO 2009/02049 SEQ ID NO:2), and a subset of the enzyme variants of Table 2 (the so called “7X variants”), as illustrated in FIG. 9 and FIG. 10; where the data illustrated therein demonstrate the thermal tolerance of the tested exemplary polypeptides (variants of the “parental” or “wild type” SEQ ID NO:6, WO 2009/02049 SEQ ID NO:2) at 96° C. through 100° C. In these figures, purified enzyme was heated for 30 minutes at the temperature indicated in the figures, and residual (thermotolerant) activity was measured at 37° C.

Heating temperatures between 84° C. and 95° C. activates the “thermal tolerant” variants (variants of the “parental” or “wild type” SEQ ID NO:2) slightly, resulting in having a residual (thermotolerant) activity of greater than the initial activity level (i.e., greater than 100%) (possibly due to improved folding upon cooling). As such, residual activity was normalized to 100%. FIG. 9 illustrates a graphic summary of data from these thermal tolerance studies for the enzymes of the invention identified as “10X-1”, “12X-1”, “13X-1”, “12X-6”, “11X-1”, “11X-2” and “7X”, in addition to wild type; and FIG. 10 is a “close-up” of part of FIG. 9.

Thus, in one aspect, the disclosed cellulases are thermotolerant and/or thermostable; for example, the enzyme can retain at least 75% residual activity (e.g., glucanase activity) after 2 minutes at 95° C.; and in another aspect, retains 100% activity after heating for 30 minutes at 95° C. In yet another aspect, the enzyme retains 100% activity after heating for 30 minutes at 96° C. 97° C. 98° C. or 99° C. In yet another aspect, the disclosed cellulases retain at least 90% activity after heating for 30 minutes at 100° C.

In some embodiments, the enzymatic hydrolysis of pNP-β-D-lactopyranoside by the disclosed cellulase can be used as a measure of activity of the enzyme. The liberation of p-nitrophenol can be followed spectrophotometrically at 405 nm. The increase in absorbance at 405 nm can be converted to μmoles of p-nitrophenol by using a standard absorbance at those defined conditions. One unit of activity is defined as the quantity of enzyme required to liberate 0.42 μmole of p-nitrophenol from 2 mM pNP-β-D-lactopyranoside during one minute at pH 7.00 and 80° C.

An improved nucleotide sequence encoding the cellulase of SEQ ID NO:2 (corresponding to WO 2009/02049 SEQ ID NO:9) was disclosed in U.S. Provisional Application No. 61/618,610 filed on Mar. 30, 2012; the entire disclosure of which is incorporated herein by reference thereto. This cellulase of SEQ ID NO:2 was evolved from the parent cellulase enzyme isolated from a DNA library originating from Thermotoga maritima strain MSB8. Enhancing expression of the disclosed cellulase involved 14 base changes with respect to the previously reported Open Reading Frame to generate SEQ ID NO:1 (below). These changes are silent as to the encoded protein. The 14 base changes are set forth below. “Position” indicates the number of the nucleotide within the Open Reading Frame, with the first nucleotide of the first codon numbered as 1. The mutation is specified using the notation (old nucleotide) (position) (new nucleotide). The mutations are as follows: T6C, T9C, T15G, A22C, G24T, A33C, A39C, A40C, A42C, A54C, A57C, T66C, G81A, A84C.

The base changes which distinguish SEQ ID NO:1 from prior reported sequences encoding the disclosed cellulase, collectively and individually, result in an Open Reading Frame which leads to a higher level of protein expression than previously employed nucleotide sequences encoding the same protein.

SEQ ID NO:1 is used to direct an increased level of expression in a number of systems in which the disclosed cellulase protein (SEQ ID NO:2) may be expressed. SEQ ID NO:1 may be introduced into any number of expression systems to express the disclosed cellulase at an improved accumulation level. For example, SEQ ID NO:1 may be introduced, either on a plasmid or stably transformed into the genome of, for example, any number of gram negative bacterial systems such as E. coli, Pseudomonas species such as fluorescens, Pseudomonas putida, Pseudomonas aeruginosa, Ralstonia species, or Caulobacter species. Similarly, SEQ ID NO:1 may be introduced into any number of gram positive bacterial expression systems such as Bacillus species such as Bacillus subtilis, Bacillus megaterium, Bacillus brevis, Lactococcus species such as Lactococcus lactis, Lactobacillus species, Streptomyces species such as Streptomyces lividans. Other gram negative, gram positive or unrelated eubacterial or archaeal expression systems may be used to express SEQ ID NO:1.

More specifically, SEQ ID NO:1 may be introduced into a plasmid to direct its expression. Plasmids which SEQ ID NO:1 may be introduced include, for example, E. coli expression vectors of the families pQE, pET, and pASK; Pseudomonas expression vectors of the families pCN51 LT8. RSF1010, pWZ112T, and pMYC; Bacillus expression vectors of the families pBAX, pHT01, and pHIS1525; Streptomyces expression vectors of the families pIJ6021 and pIJ2460; and Lactococcus: expression vectors of the families pNZ9530 and pNZ8148, for example. These examples are for demonstrative purposes and do not represent a complete set of vectors in which the polynucleotide sequence of SEQ ID NO:1 can be expressed.

### Viscosifiers and Other Additives

In some embodiments, the polymeric viscosifier may be selected from one or more of guar, crosslinked guar, hydroxypropyl guar, carboxymethyl guar, carboxymethyl hydroxypropyl guar, carboxymethyl cellulose, barley β-glucan, and locust bean gum. In some embodiments, the hydrolytic pattern of the disclosed cellulases is both endo and exo, allowing effective reduction in the viscosity of polysaccharides, e.g., guar and derivatized guar solutions, by cleaving within long polysaccharide chains and also by cleaving disaccharide units from the ends of the polymers. Besides the aforementioned polysaccharides, other substrates of the disclosed enzymes include those capable of forming linear or crosslinked gels (e.g., borate crosslinked guar). Examples of suitable polysaccharide substrates include galactomannan gums, guars, derivatized guars, cellulose and cellulose derivatives, starch, starch derivatizes, xanthan, derivatized xanthan and mixtures thereof. Specific examples also include, but are not limited to, guar gum, guar gum derivative, locust bean gum, karaya gum, xanthan gum, cellulose and cellulose derivatives, etc. Typical polymeric viscosifiers or gelling agents to which the disclosed enzymes may be directed include guar gum, hydroxypropyl guar, carboxymethyl hydroxypropyl guar, hydroxyethyl cellulose, carboxymethyl hydroxyethyl cellulose, carboxymethyl cellulose, dialkyl carboxymethyl cellulose, etc. Other examples of polymers include, but are not limited to, phosphomannons, scerolglucons, dextrans and other types of polymers. In some embodiments, a polymer substrate is carboxymethyl hydroxypropyl guar. In some embodiments, a disclosed enzyme may also be effective in hydrolyzing biogums (e.g., succinoglycan biogums made from date syrup or sucrose). In some embodiments, a disclosed enzyme may be used to hydrolyze cellulose-containing or derivatized cellulose-containing polymers—typically, the enzymes attack glucosidic linkages of the cellulose backbone. The disclosed enzymes may be suitable for degrading the polymer into mostly monosaccharide units, in some cases, by specifically hydrolyzing the exo(1,4)-β-D-glucosidic and endo(1,4)-β-D-glucosidic linkages between monosaccharide units and the cellulose backbone in the (1,4)-β-D-glucosidic linkages of any cellobiose fragments.

The fracturing fluid composition described herein may also comprise additional components, including agents to control fluid loss, reduce formation damage, adjust and/or buffer pH, control microbial growth and improve temperature stability. Crosslinkers may include borate, titanate, zirconate, and aluminum compounds. Typical hydraulic fracturing mixtures include about 98% water and sand and about 2% other additives, selected from acid, anti-bacterial agents, breaker (enzyme or chemical/oxidative), clay stabilizer, corrosion inhibitor, crosslinker, friction reducer, gelling agent (viscosifier), iron control, pH adjusting agent, scale inhibitor and surfactant.

### Performance of Disclosed Cellulases in Fracturing Fluids

In each fracturing job that uses the disclosed cellulase, field operators will first perform an enzyme dose optimization study in an industrial lab. The disclosed cellulase will be diluted to a concentration of 10˜400 ppm and mixed with the polysaccharide gel forming compound, e.g., linear or cross-linked guar gum (25-60 lb/1,000 gal). Depending on the application conditions, guar gum or derivatized guar is often cross-linked using a borate cross-linker, especially for wells where higher temperature, pressure, and pH conditions are present. The enzyme dose information resulting from such optimization study will be used in the actual fracturing job.

The unique activity profile of the disclosed cellulases allow for complete hydrolysis of guar-based fracturing fluids in a smooth and controlled manner in deep wells, where high temperature and high pH conditions are present. Compared to chemical breakers, the disclosed cellulase of the present invention provides a non-corrosive and environmentally benign alternative to the harsh and non-selective chemical breakers.

Enzyme breakers have been used for hydrolyzing polysaccharide viscosifiers, including e.g., guar gels at temperatures below 150° F., for many years. There is, however, an industry-wide demand for enzyme breakers that can function under higher temperature (e.g., 200-250° F.) and extreme pH (≥10.5) conditions. To address this demand, the inventors have developed fracturing fluids comprising an exceptionally thermo-stable cellulase. See also PCT publication WO 2009/02049, which describes inter alia the protein and the DNA encoding the protein. The disclosed cellulases evolved from the parent enzyme have been shown to exhibit well differentiated performance under the extreme down-hole conditions encountered in gas shales and deeper oil/gas wells.

The disclosed cellulases can effectively break linear and borate cross-linked guar under broad ranges of temperature (80° F. up to 250° F.) and pH (3.0 up to 10.5). The results of rheological tests show that only a small dose is required (100 ppm or less) to achieve the complete break. The enzymatic reaction can be triggered by the changes of temperature and pH during fracturing operations. The disclosed cellulases also exhibit an excellent dose response that allows the operator to generate an ideal viscosity-time profile by adjusting enzyme dosage. The dose-dependent behavior will prove highly beneficial as it avoids premature viscosity loss and undesirable proppant screen-outs. Even in the presence of fluid additives, such as buffers, salts, stabilizers, crosslinkers, etc., the disclosed cellulases remain active for effective viscosity reduction.

The disclosed cellulase breakers reduce gel viscosity by specifically targeting β-1,4 glycosidic bonds between the mannose units in guar. Carbohydrate profiling tests demonstrated that this enzyme effectively and efficiently breaks the long guar polymers into small, soluble fragments that will eliminate gel re-healing. The conductivity tests demonstrate the complete hydrolysis of guar and removal of polymer residues that cause formation damage and reduce well conductivity.

As a biocatalyst for a guar breaking reaction, the disclosed cellulases, which exhibit mannanase activity, have several significant advantages over traditional oxidative chemical breakers. First, the enzyme specifically breaks long chain guar gum polymer through endo or exo-beta mannanase activity without undesirable reactions to the wellbore, formation or fracturing equipment. Second, the enzyme is not consumed in the reaction and can continue working on other guar polymers during their life time, thus providing an extended and controlled breaking profile. Third, the enzyme can break guar polymers into much smaller fragments, thus providing a more complete guar break with less residue. Consequently, efforts have been made by scientists to discover and characterize hyper-thermostable cellulases from extreme natural environments. Thermotoga maritima sp., a hyperthermophilic bacterium identified from a hydrothermal vent sample, has a cellulase with endo-mannanase activity that can specifically cleave β-1,4 bond linked polysaccharides of long chain guar gum. Mathur E J, Lam D E, “Carboxymethyl cellulase from Thermotoga maritima”, U.S. Pat. No. 5,962,258 (1999), U.S. Pat. No. 6,008,032 (1999) and U.S. Pat. No. 6,245,547 (2001). Specifically, this enzyme demonstrates a temperature optimum of 180° F., which is significantly higher than other known endo-mannnases in the glycoside hydrolase family. Pereira J H, Chen Z W, McAndrew R P, Sapra R, Chhabra S R, Sale K L Simmons B A, Adams P D, “Biochemical characterization and crystal structure of endoglucanase Cel5A from the hyperthermophilic Thermotoga maritima”, Journal of Structure Biology, 2010, 172; 372-379; Wu T H, Huang C H, Ko T P, Lai H L, Ma Y H, Chen C C, Cheng Y S, Liu J R, Gup R T, “Diverse substrate recognition mechanism revealed by Thermotoga maritima Cel5A structures in complex with cellotetraose, cellobiose and mannotriose”, Biochimica et Biophysica Acta, 2011, 1814; 1832-1840.

Thermostability of Cellulase Evaluated with Differential Scanning Calorimetry (DSC)

Enzymes have 3-dimensional structure which is critical for its catalytic function. Under certain conditions (heat, denaturant, extreme pH), enzymes can become unfolded and lose their catalytically active 3-dimensional structure. The melting temperature (Tm) is the temperature at which 50% of a protein becomes unfolded. This parameter is widely used for evaluation of thermostability of enzymes or proteins. Tm can be measured by differential scanning calorimetry (DSC).

DSC tests were carried out with cellulase wild-type protein and the selected cellulase variant. All protein samples were analyzed at a concentration of 1 mg/ml and a scan rate of 1° C./min. The temperature range of each scan was 160˜250° F. A constant pressure of 4.6 atm was maintained during all DSC experiments to prevent possible degassing of the solution on heating. Tm was calculated with the available software package. FIG. 7 show the Tm results of wild-type and a selected cellulase variant. There is a 24° F. increase of Tm value observed with the final cellulase candidate as compared to the wild-type enzyme. The Tin is pH dependent. The Tm difference between variant and wild-type is 24° F. at pH 6.5 while the difference is 13° F. at pH 10.5.

### Temperature Profiling of the Cellulase Variant Using a Chemical Surrogate

The cellulase variant was further evaluated at various temperatures for its 1-4 linkage cleavage activity. The molecule pNP-β-D-lactopyranoside (FIG. 8A) was used as a surrogate substrate. The enzymatic hydrolysis of pNP-β-D-Lactopyranoside by cellulase can produce free p-Nitrophenol, which can be measured spectrophotometrically at 405 nm. FIG. 8B exhibits the temperature profile of the cellulase variant up to 194° F., which is the temperature limit for the spectrophotometer. As shown in FIG. 8B, this cellulase variant has an optimal temperature at 180° F.

In order to evaluate cellulase activity at above boiling temperature (>212° F.), an assay was developed to quantitatively measure the residual activity of the cellulase variant. The enzyme was mixed with 25 lb guar per 1000 gal (ppt) at 1:1 ratio before heat challenged at 225° F., 250° F., and 275° F. for 10, 20, and 30 minutes, respectively. Subsequently, activity of the heat challenged enzyme was measured using the pNP absorbance assay by the addition of 30 uL of heat treated enzyme to 3000 uL of 2 mM pNP-B-D-lactopyranoside, followed by measuring 405 absorbance for 10 minutes. FIG. 8C shows the residual activity of the cellulase variant at 225° F. 250° F., and 275′ F. Robust cellulase activity was observed at 225° F. when the enzyme was heat-treated for 10-30 minutes. When heat-treated cellulase was applied to borate crosslinked guar (25 lb per 1000 gal), complete gel breaks were observed at a small dose (20 gpt). The cellulase activity becomes compromised at 250° F., and 275° F. with relative residual activity at 11-14% of that at 225° F. (FIG. 8C), suggesting a small percentage of cellulase can still perform measurable catalytic reaction even after high temperature (>250° F.) treatment.

### pH Profiling of the Cellulase Variant Using a Fluorescent Surrogate

The molecule 4-Methylumbelliferyl β-D-cellobioside is a surrogate substrate that can be cleaved by cellulase at higher pH conditions (FIG. 8A). The cleavage product, 4-methylumbelliferone can be quantified fluorometrically using excitation and emission wavelengths of 365 nm and 455 nm. Two millimoles of 4-Methylumbelliferyl β-D-cellobioside were dissolved in a buffer system with pH values ranging from 9.5 to 10.5. Cellulase activity at different pH conditions was measured kinetically in a spectrophotometer by addition of 30 uL cellulase into 3000 uL of 2 mM 4-Methylumbelliferyl β-D-cellobioside. The slope of each reaction curve was obtained by linear curve fitting. The relative activity was calculated according to results obtained at pH 9.5. At pH 10.5, a pH condition that is highly relevant to borate crosslinked guar fluids, cellulase exhibits reasonable activity (FIG. 9).

### Confirmation of Enzymatic Breaking of β-1,4-Linkage but not α-1,6-Linkage Polysaccharides by HPLC

Guar, a long-chain polysaccharide composed of mannose and galactose sugars, is the major viscosifier in water based fracturing operations. Polymannose forms a long chain backbone through β-1,4-linkage while galactose unit is attached to the mannose unit through α-1,6 linkage. The ratio of mannose to galactose sugars may be ranging from 1.6:1 to 1.8:1. Importantly, the polymannose backbone of guar is not soluble in water and galactose branches significantly increase water solubility. However, it has been reported that as few as 6 contiguous un-branched mannose units can form a local helical structure which is completely insoluble (11). Therefore, if cellulase breaks at the α-1,6 linkage of guar molecule, significant amount of insoluble residue can be produced. As a result, the conductivity of the proppant pack can be impaired.

In order to confirm that enzymatic activity of the cellulase variant targets specifically at β-1, 4 glycosidic bonds of polysaccharide, a normal phase HPLC method was adopted for analysis of two surrogate substrates, 1,4-β-D-Mannopentaose and 63,64-α-D-Galactosyl-mannopentaose (FIG. 10A). The cellulase variant at 3 units/mL was incubated with 1 mg/ml, substrates for one hour at 80° C. at pH 7.0. Using an amide column, actonitrile/H2O/triethylamine as a mobile phase, and an ELSD detector, the baseline separation for mannan oligosaccharides by HPLC was obtained. FIG. 10B illustrates HPLC elution profiles of different mannan oligosaccarides including M1, M2, M3, M4, M6, and galactose standards (M1-M6 indicates mannan oligosaccharide chain length). FIG. 4C shows an example of how β-1,4-D-Mannopentaose, a mannan oligosaccharide with only β-1,4 linkage in its structure, was digested by the cellulase variant. HPLC profiles demonstrate β-1,4-linkage cleavage with preferable production of M3 oligosaccarides along with production of M1, M2 and M4, confirming that the cellulase variant specifically attacks β-1,4 linkage when breaking down the mannan oligosaccharides (FIG. 10C(a)-(b)). On the other hand, for 63,64-α-D-Galactosyl-mannopentaose, a compound containing both a β-1,4-linkage and α-1,6-linkage, the HPLC profiles show that cellulase variant only breaks the β-1-4 linkages and produces an OGGM4 peak and a single unit mannose peak (FIG. 10C(a)). Using galactose as a reference, no peak was observed for the single unit galactose from enzyme treated 6³,6⁴-α-D-Galactosyl-mannopentaose, confirming that the α-1, 6 linkages remain intact after mananase treatment (FIG. 10C(b)).

Preparation of Crosslinked Guar Fluids with Breakers

Linear guar fluids were prepared by adding the dry polymer to water that was stirred in a blender sufficiently to generate a vortex. Stirring was continued for about 30 minutes for complete hydration. The pH was adjusted with a solution of sodium hydroxide. For crosslinked fluids, clay stabilizer and cleanup surfactant, when used, were added after full hydration of the guar. Next, caustic was added, followed by the enzyme addition. The crosslinkers were added last. The borate crosslinker was delayed in action by slow dissolution of the active boron species. The zirconium crosslinker was also delayed due to the presence of sesquicarbonate buffer used at 6 ppt.

### Bottle Testing

Jars of guar or crosslinked guar were prepared with either ammonium persulfate (APS) or cellulase breakers. The jars were aged in a water bath and the level of breaking was visually assessed by tilting the bottles and observing the fluid movement. The bottle tests allowed a quick method for evaluating the effect of concentration, pH and temperature on breaking of a given fracturing fluid. Because the tests are qualitative in nature, the results are not shown.

### Rheology

The viscometer measures viscosity using a cup and bob technique (Rotor 1 and Bob 5) with model 50 specifications under a nitrogen pressure of 400 psi. Fluid temperature was increased to the desired value by an oil bath. Heating takes about 20 minutes to reach the desired temperature and that temperature was maintained for the test duration. Continuous measurement of viscosity occurs at a rotational speed that delivers a wall shear rate of 100 s-1 with periodic shear ramps at 100, 75, 50, 25, 50, 75 and 100 s-1. The shear ramp data is used to calculate power law parameters (n′ and K′) for prediction of viscosity at lower shear rates that are commonly found in a fracture.

FIG. 11 demonstrates a range of activity in breaking 80 lb per 1000 gallon linear guar solution at 180° F. The concentration of cellulase was kept constant at 200 ppm. As pH increased from 9 to 11, a noticeable delay in breaking was observed, consistent with a lower activity for the enzyme at elevated levels of pH. Even at a pH of 11, the solution shows a slow breaking effect compared to the control that lacks enzyme.

FIG. 12 shows the effect of the enzyme concentration on breaking for a borate-crosslinked guar fluid at 200° F. and pH of 10.5. Increasing dosages of cellulase result in more significant breaking of viscosity. The rheology results were run for several hours to show the continual loss of viscosity with time. Clearly for crosslinked gels, more enzyme is needed versus the linear gels containing a higher concentration of guar. The viscosity of crosslinked fluid with a level of 50 ppm cellulase was indistinguishable from that of the crosslinked fluid control whereas higher levels of enzyme did show breaking activity.

Importantly, the enzyme is still active at 225° F. and pH of 10 when thermal stabilizer is included at a level of 10 ppt (FIG. 13). The control shows a gradual thermal deterioration but the enzyme clearly accelerates the degradation of viscosity. FIG. 14 presents comparison data of cellulase and encapsulated ammonium persulfate at 225° F., and pH of 10 for a zirconium-crosslinked fluid using carboxymethylhydroxyethylcellulose (CMHPG). A concentration of 100 ppm provides a similar break profile to ammonium persulfate while 200 ppm results in immediate loss of viscosity. Note that the encapsulated ammonium persulfate releases some of the active material via thermal degradation of and/or diffusion through the encapsulating material which results in delayed breaking.

### Residue Analysis

Jars of guar or crosslinked guar were prepared with either ammonium persulfate or cellulase breakers. The jars were aged in a water bath overnight at 180° F. The contents were centrifuged at 3000 rpm for 5 minutes and vacuum filtered through a nominal 5 micron, weighed paper. The paper was dried overnight at 110° F., and reweighed to calculate the amount of residue recovered. This value is expressed as a percentage of the original weight of polymer in the solution. The filter paper plus residue was then dried for another 16 hours and reweighed to ensure the moisture had been removed. However, the data suggest that the additional 16 hours of drying time is seen to be unnecessary. The fluids tested included 80 ppt of linear guar at pH of 10. Cellulase was tested at 200 and 400 ppm while ammonium persulfate was used at 1 and 5 ppt.

The results show that surprisingly the solutions containing cellulase had lower amounts of residue than the corresponding solutions with ammonium persulfate (FIG. 15). In contrast, the oxidative breakers appear to cleave more randomly and result in a higher level of insoluble fragments that are captured as residue. If the jars were left for a longer time period at temperature (i.e., 180° F.), it is possible that the residue will decrease even further for the enzyme treated fluids but not for the fluids containing oxidative breaker. For ammonium persulfate, the residue is sensitive to breaker concentration, whereas the sensitivity to enzyme concentration is much lower.

### Conductivity Study

Fracture conductivity was measured using a modified API cell with a fixed bottom piston. Conditions were 180° F., 3000 psi closure stress, 2 lbm/ft2 proppant loading (20/40 mesh Ottawa sand) and about a 5× concentration of fracturing fluid following leakoff. The fluid was left at temperature overnight before four hours of cleanup followed by a minimum of one hour of pack flow. The cleanup alternates flow through the pack and through the core each hour using 2 wt % KCl solution. Measurement of final conductivity, stable within 4% for one hour, was performed using 2 wt % KCl at a rate of 3 mL/min. Retained conductivity was calculated by comparison with the conductivity measured for the same conditions using 2 wt % KCl in place of the fracturing fluid.

Linear guar and boron-crosslinked guar with ammonium persulfate or cellulase were run as well as one test with crosslinked guar using both breakers. For linear guar, higher levels of retained conductivity were achieved with cellulase than with ammonium persulfate. Similarly, encapsulated ammonium persulfate provided a lower retained conductivity than did 200 ppm of cellulase for crosslinked-borate fluid (FIG. 16). The combination of cellulase and encapsulated persulfate had even higher retained conductivity than either one of the breakers alone. However, the highest result was obtained with a 400 ppm dosage of the enzyme. Since the conductivity test is shut-in overnight, the cellulase is allowed extra time for reducing molecular weight of the guar. This will directly result in enhanced conductivity for the proppant pack.

In summary, the evolved cellulase variant is compatible with various additives in borate crosslinked guar gel fluids. Particularly, this cellulase variant shows robust guar gel breaking profiles in the temperature range of 180-225° F., and a pH range up to 10.5. The pH limitation prevents testing this enzyme with borate crosslinked guar fluids in higher temperature range. However, positive guar breaking profiles were observed with zirconium crosslinked CMHPG at temperature of 225° F. Notably, residue analysis demonstrates that mannase treated fluids produce significantly less residue than ammonium persulfate treated fluids. More importantly, cellulase treatment achieved significantly higher retained conductivity than did ammonium persulfate treatment, confirming the notion that enzymes can provide a more complete guar break and improved well conditions relative to chemical oxidizers. Moreover, use of cellulase is compatible with addition of ammonium persulfate should that be useful in certain oilfield applications.

### DESCRIPTION OF THE FIGURES

FIG. 1 is an image of SDS PAGE gel electrophoresis displaying various level of protein expression, as described in Example 2.

FIG. 2 is a bar graph showing the level of activity of protein preparations, as described in Example 3.

FIG. 3 is SEQ ID NO:1, the polynucleotide with 14 silent mutations: T6C, T9C, T15G, A22C, G24T, A33C, A39C, A40C, A42C, A54C, A57C, T66C, G81A, A84C, as compared to SEQ ID NO:3.

FIG. 4 is SEQ ID NO:2, the polypeptide encoded by SEQ ID NO:1, 3, and 4.

FIG. 5 is SEQ ID NO:3, the unmodified parent polynucleotide sequence of SEQ ID NO:1 and 4.

FIG. 6 is SEQ ID NO:4, the polynucleotide with 14 silent mutations: T6C, T9C, T15G, A22C, G24T, A33C, A39C, A40C, A42C, A54C, A57C, T66C, G81A, A84C, as compared to SEQ ID NO:3, plus one additional point mutation upstream from the start codon (additional upstream sequence shown).

FIG. 7. DSC tests of the wild type cellulase and the selected cellulase variant were performed with calculated Tm value labeled. A 24° F. increase of Tm was observed with the cellulase variant as compared to the wild type cellulase.

FIG. 8A. Chemical structure of pNP-β-D-lactopyranoside.

FIG. 8B. Temperature profiling of cellulase measured by an pNP absorbance assay (405 nm) at different temperature (from 70° F. to 200° F.). The activity was calculated as percentage of activity at 180° F. The activity at above boiling temperature (>212° F.) can be further evaluated with residual activity assay (FIG. 8C).

FIG. 8C. Residual activities of the cellulase variant were evaluated using heat challenged samples. The relative activity was calculated as percentage of activity obtained at 225° F.

FIG. 9, pH profiling of the cellulase variant using a fluorescent assay. Relative activity was calculated by the percentage of activity at pH 9.5.

FIG. 10A. Structures of two compounds for HPLC study. 1,4-β-D-Mannopentaose is a compound with 5 units of mannose connected through 1,4-β linkage. 6³,6⁴-α-D-Galactosyl-Mannopentaose is a compound mimic for guar gum structure with two galactose units connected through α-1,6 linkages to 1,4-β-D-Mannopentanose, which allows analysis of hydrolytic products from cellulase treatment.

FIG. 10B. HPLC analysis of oligosaccharides standards (M1-M6, and galactose). Note the mannose single unit (M1, red peak) is well separated from single unit galactose (blue peak) in the elution profile of HPLC.

FIG. 10C. HPLC analysis of hydrolytic products from 1,4-β-D-Mannopentaose and 6³,6⁴-α-D-Galactosyl-Mannopentaose after mannose treatment.

a) enzymatic digestion of 1,4-β-D-Mannopentaose. HPLC elution profile of control sample with no enzyme treatment is shown in red while cellulase variant treated 1,4-β-D-Mannopentaose is shown in blue.
b) enzymatic digestion of 63,64-ÿ-D-Galactosyl-Mannopentaose. O-GGM5 represents 6³,6⁴-α-D-Galactosyl-Mannopentaose with cellulase treated sample shown in red and no enzyme sample shown in blue. Single unit mannose (shown in pink) and galactose (shown in green) are included as references. Note there is not a galactose peak but only a mannose peak in the HPLC elution profile of cellulase treated O-GGM5, confirming that the cellulase variant specifically cleaves β-1,4 glycosidic bonds between the mannose units.

FIG. 11. Effects of pH on viscosity of 80 lbm guar/1000 gal US at 180° F. Shear ramps have been removed for clarity.

FIG. 12. Rheology study of the cellulase variant on borate crosslinked guar at 200° F. with pH=10.5. All fluids contained a clay stabilizer and surfactant and used a delayed crosslinker.

FIG. 13. Rheology study of the cellulase variant on borate crosslinked guar at 225° F. with pH=10. All fluids contained 10 lbm thermal stabilizer/1000 gal US and used a delayed crosslinker.

FIG. 14. Rheology study of the cellulase variant with a zirconium crosslinked CMHPG at 225′ F and pH=9.9 with 10 lbm thermal stabilizer/1000 gal US.

FIG. 15. Residue analysis comparing ammonium, persulfate with cellulase. Higher breaker levels reduce the residue in both cases, but the oxidative breaker shows higher amounts of residue.

FIG. 16. Comparison of conductivity results for cellulase versus ammonium persulfate for both linear and boron-crosslinked guar formulations. Linear: 80 lbm guar/1000 gal US with cellulase or live ammonium persulfate; Crosslinked: 30 lbm guar/1000 gal US with cellulase or encapsulated ammonium persulfate.

### DEFINITION OF TERMS

“cellulase” are enzymes having cellulase, endoglucanase, cellobiohydrolase, beta-glucosidase, xylanase, mannanase, β-xylosidase, arabinofuranosidase, and/or oligomerase activity.

“cellulolytic activity” is an enzyme having cellulase, endoglucanase, cellobiohydrolase, beta-glucosidase, xylanase, mannanase, β-xylosidase, arabinofuranosidase, and/or oligomerase activity.

A “codon” is a three polynucleotide sequence that specifies the identity of an amino acid to be added to a protein.

A “silent mutation” is a mutation in a codon that does not result in the specification of a different amino acid.

An “Open Reading Frame” is a series of codons that specifies the sequence of amino acids in a protein.

A base “position” is the numerical location of a base in a polynucleotide sequence, counted consecutively from the start of the open reading frame or from some other reference marker.

To “encode” a protein means to specify the amino acid sequence of that protein.

A “mutation” is a change in a nucleotide sequence or an amino acid sequence compared to a reference.

A “nucleotide” refers to one of the four bases which comprise DNA sequence—Adenine (A), Thymidine (T), Guanidine (G), and Cytosine (C).

Thermotoga maritima genomic sequence” refers to the Thermotoga maritima strain MSB8 genomic sequence specified by GenBank Accession No. AE000512.

An “Expression level” for a given protein is the amount of protein generated by an expression system, such as a transformed cell culture as measured per unit volume of cell culture.

An “Expression level” for a given enzyme is the amount of enzyme activity generated by an expression system, such as a transformed cell culture as measured per unit volume of cell culture.

“Wild-type” refers to a protein or nucleic acid sequence that can be obtained in nature.

### Example 1—Method of Making Enhanced Expression Variants

Two variants (SEQ ID NO:1 and NO: 4) were designed based on SEQ ID NO:3 to mutate at the DNA level to improve the gene expression. The design takes into account of many factors that may influence gene expression. The mutations were introduced on the PCR primers using PCR techniques known to those of skill in the art. Both genes were PCR-amplified and cloned into the Pseudomonas vector pDOW1169 (DOW AgroSciences, IN) using standard molecular cloning techniques. The resulting expression constructs were transformed into Pseudomonas fluorescens DC454 (DOW AgroSciences, IN). A transformant with the SEQ ID NO:1 was designated as the lead as it showed the most enhanced expression.

### Example 2—Using SDS-PAGE Gel Electrophoresis and Nonspecific Protein Staining to Visualize Expression Levels of the SEQ ID NO:2 Polypeptide Expressed by Constructs Comprising SEQ ID NOs: 1, 3, and 4

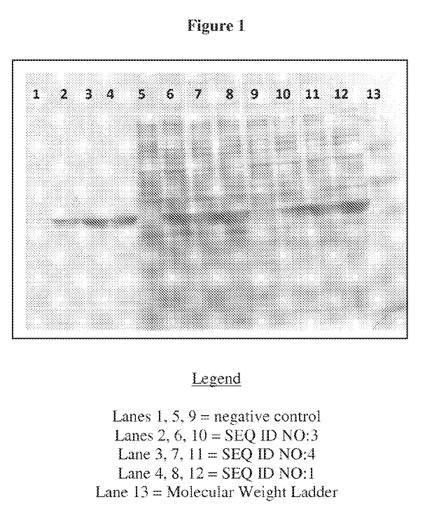

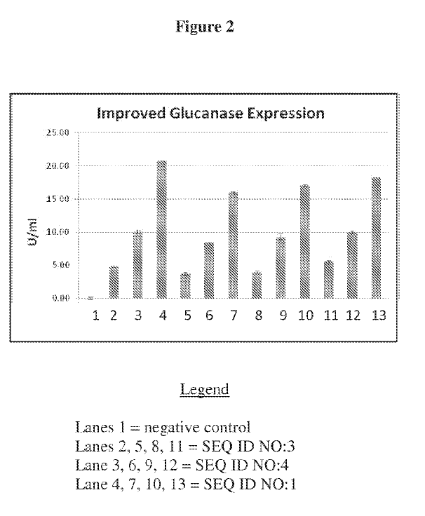

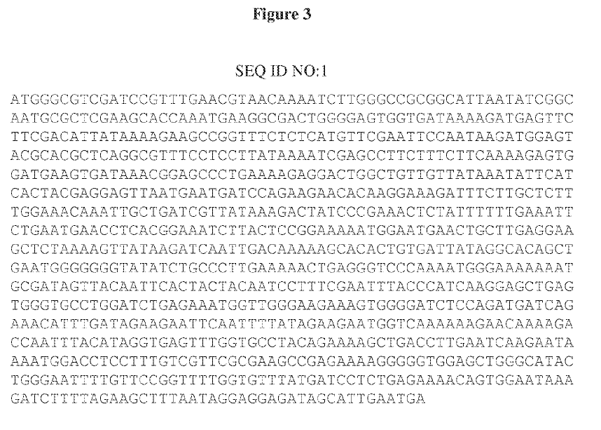

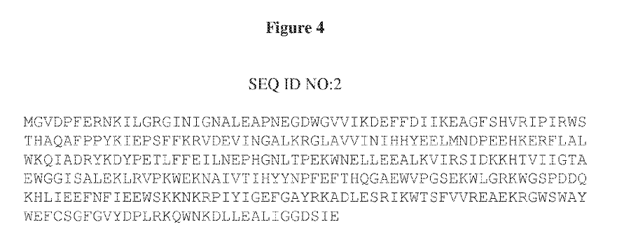

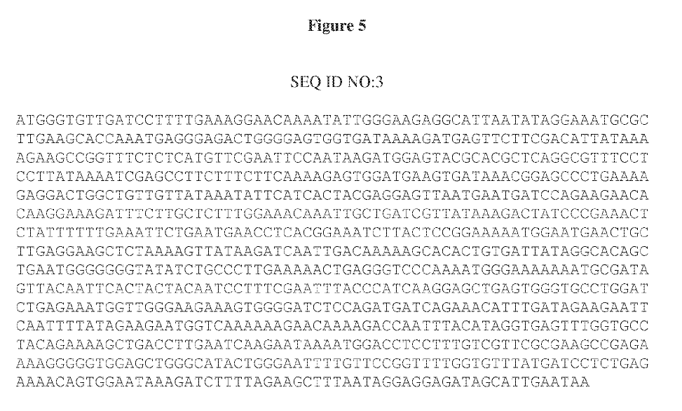

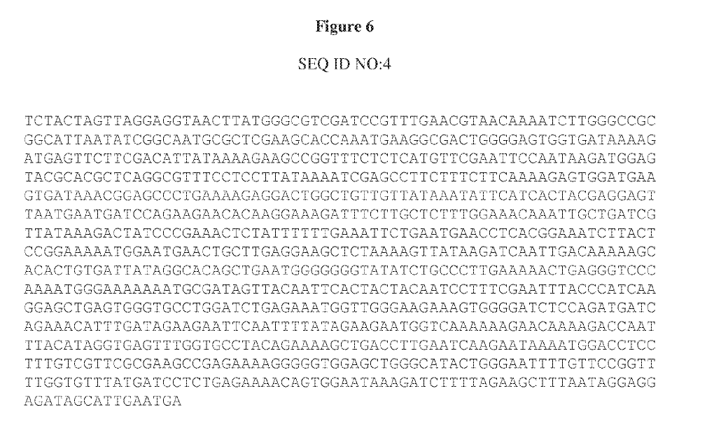

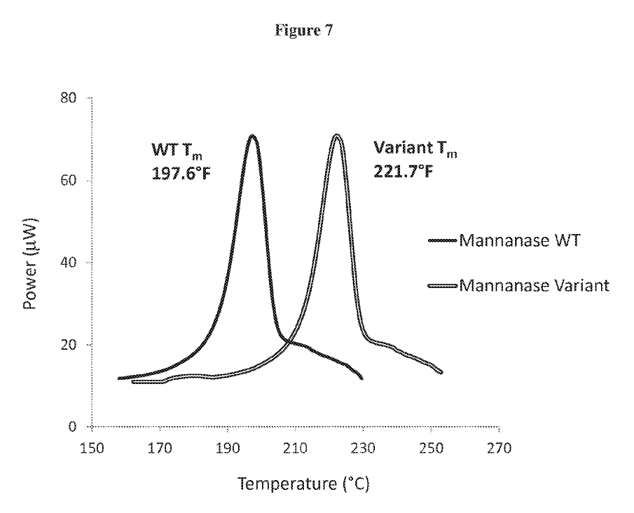

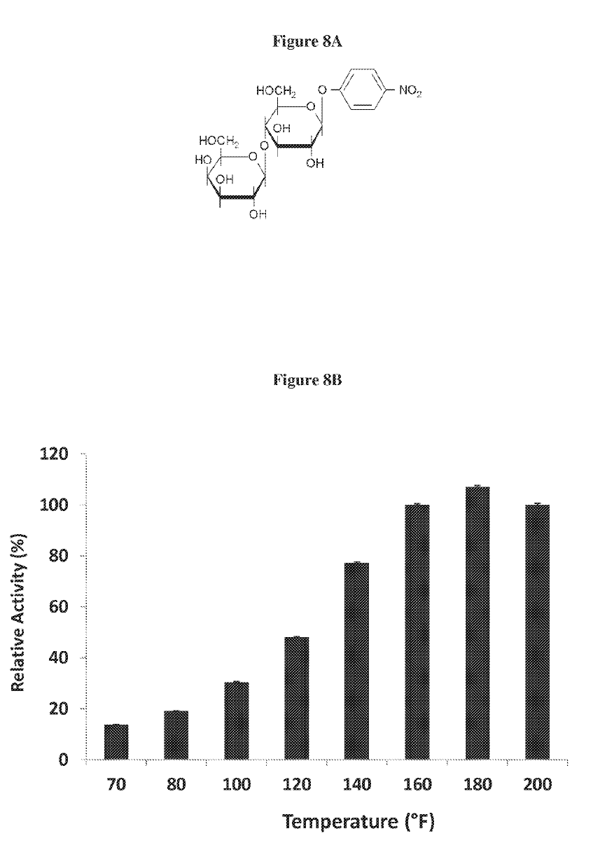

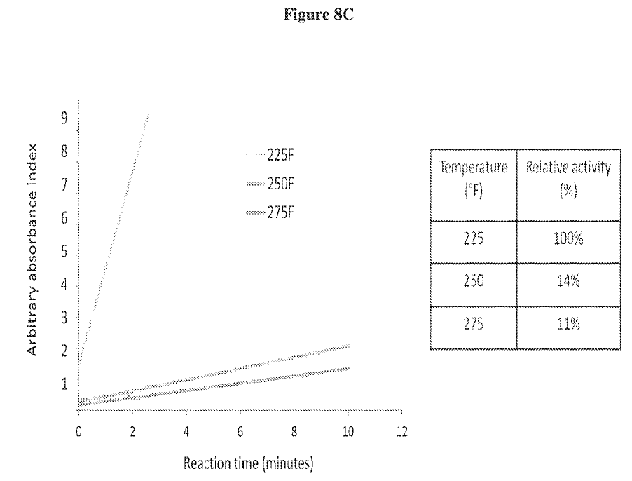

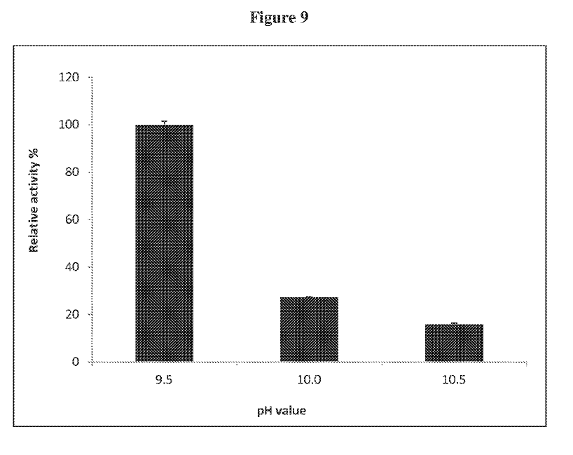

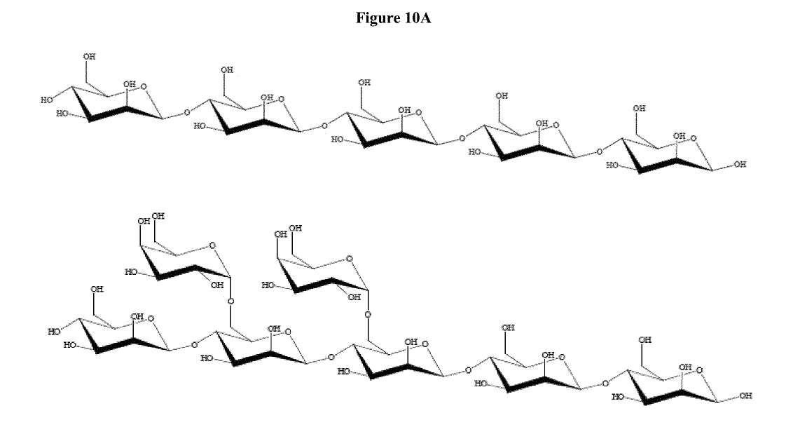

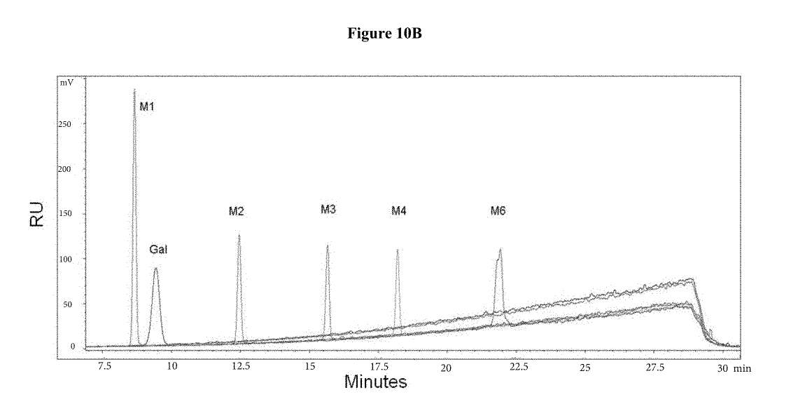

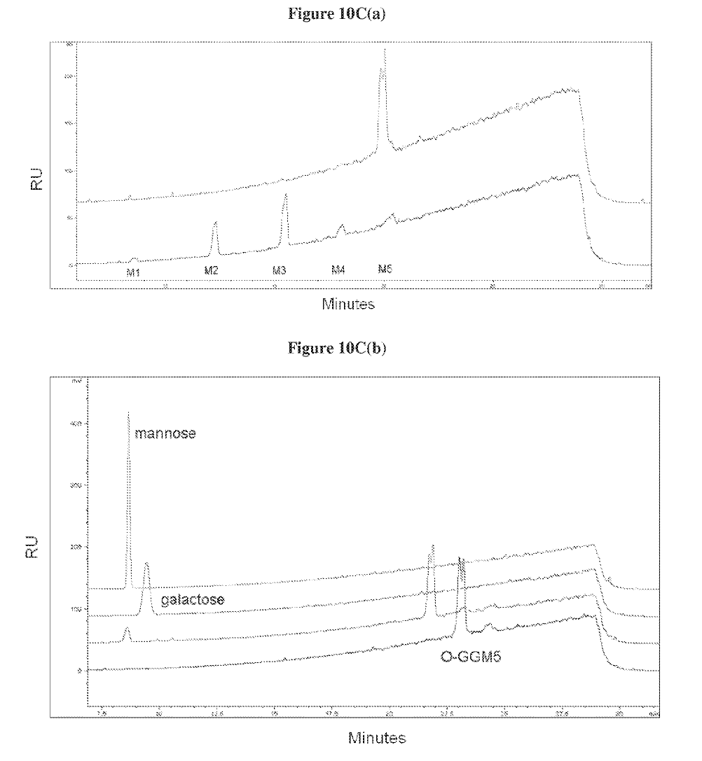

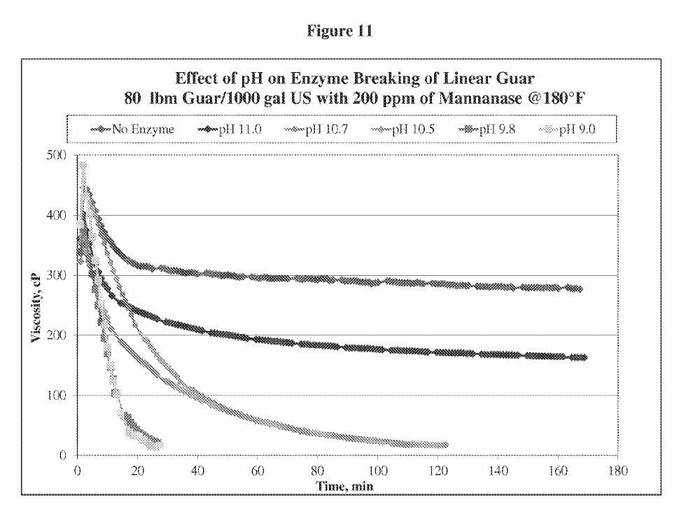

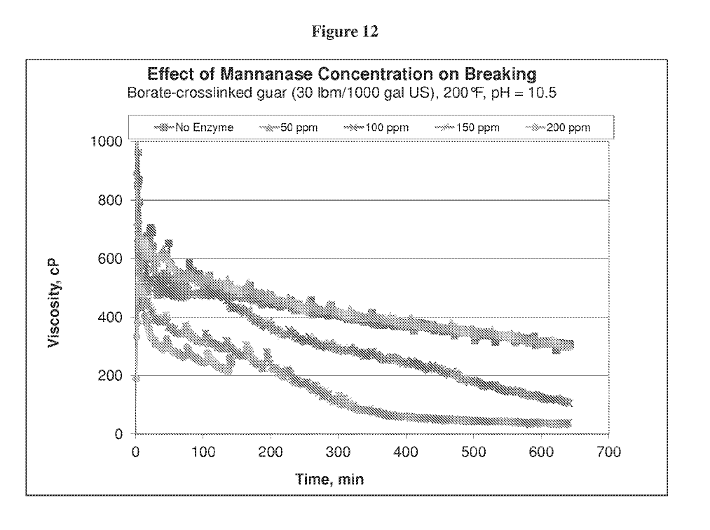

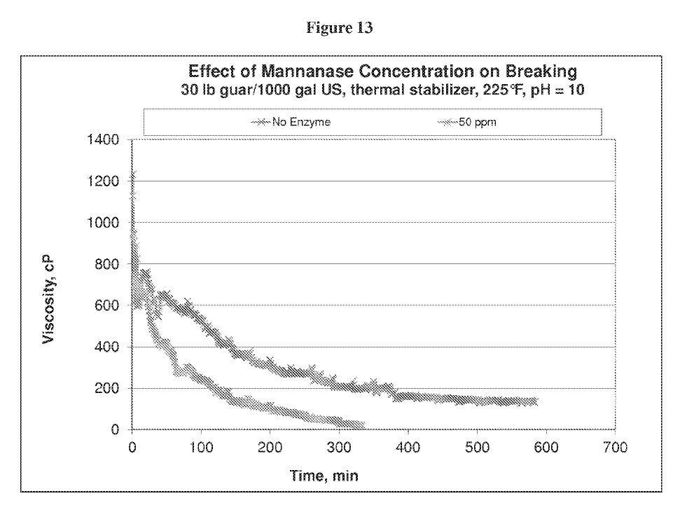

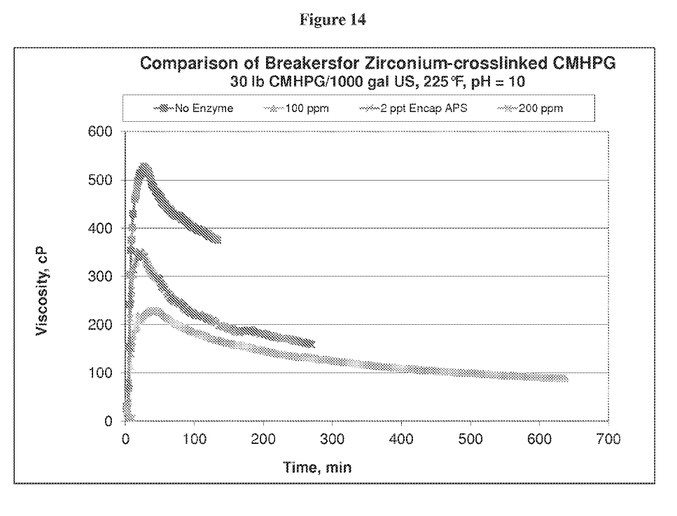

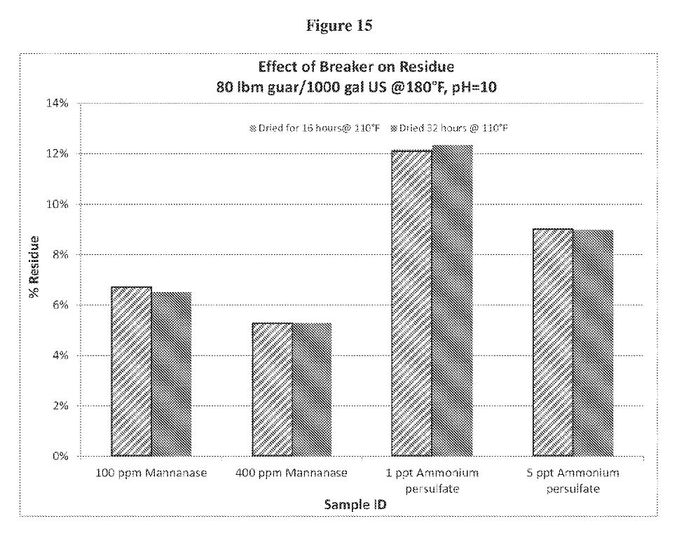

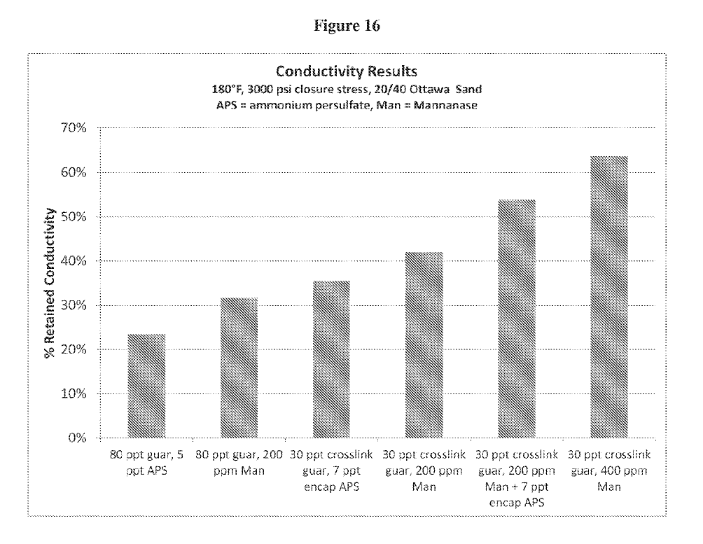

Criterion™ precast Tris-HCl polyacrylamide gel (Bio-rad Laboratories, Inc.) was used to separate proteins. The gel was run at 150V using Tris-glycine buffer (see FIG. 1). Protein loading was normalized to load proteins from 0.33 OD₆₀₀ cells for each lane. SeeBlue® pre-stained protein standard was used (Life Technologies). The gel was stained with a nonspecific dye, and each lane was visually inspected for the presence of a band at the size of SEQ ID NO:2, about 37 kilodaltons.

1.-44. (canceled)

45. A composition comprising
a polymeric viscosifier, and
an enzyme breaker comprising a wild-type cellulase derived from a hyperthermophilic bacterium or a mutated variant thereof.

46. The composition of claim 45, comprising a surfactant.

47. The composition of claim 45, comprising a thermostabilizer.

48. The composition of claim 45, wherein the polymeric viscosifier is a guar gel comprising a linear guar, a crosslinked guar, or mixtures thereof.

49. The composition of claim 48, wherein the enzyme breaker specifically hydrolyzes β-1,4 glycosidic bonds in the guar gel.

50. The composition of claim 48, wherein the enzyme breaker does not specifically hydrolyze α-1,6 glycosidic bonds in the guar gel.

51. The composition of claim 48, wherein the enzyme breaker retains its ability to hydrolyze β-1,4 glycosidic bonds in the guar gel at temperatures up to about 275° F.

52. The composition of claim 48, wherein the enzyme breaker retains its ability to hydrolyze β-1,4 glycosidic bonds in the guar gel at a pH of up to about 11.

53. The composition of claim 45, wherein the enzyme breaker is:
(a) a mutated cellulase comprising 12 mutations relative to the wild type cellulase;
(b) a mutated cellulase comprising at least one mutation selected from F38Y, Y61Q, M69E, D70P, R71S, 194Q, I166V, S183R, S191A, E212P, L231V, M276A, E277S, R280G, T297P, T301Q, and any combination thereof;
(c) a cellulase derived from Thermotoga maritima comprising at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(d) a cellulase encoded by a nucleotide sequence of SEQ ID NO:3 comprising at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(e) a cellulase encoded by a nucleotide sequence from Thermotoga maritima having at least one mutation which increases the expression level of a protein encoded by said nucleotide sequence compared to a Thermotoga maritima genomic sequence;
(f) a cellulase encoded by a first nucleotide sequence encoding the polypeptide of SEQ ID NO:2 wherein said first nucleotide sequence has been mutated with respect to a second sequence encoding SEQ ID NO:2 such that the expression level of said cellulase is increased relative to that of a protein encoded by said second nucleotide sequence;
(g) a cellulase encoded by a nucleotide sequence encoding a protein at least 50%, 60%, 70%, 80%, 90%, 95%, 97%, 98%, 99% or 100% identical to SEQ ID NO:2, or a fragment thereof, wherein said nucleotide sequence comprises at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(h) a cellulase comprising the amino acid sequence of SEQ ID NO:6, SEQ ID NO:9, SEQ ID NO:11, SEQ ID NO:13, SEQ ID NO:15, SEQ ID NO:17, SEQ ID NO:19, or SEQ ID NO:21; or
(i) a cellulase encoded by the nucleic acid sequence of SEQ ID NO:4, SEQ ID NO:5, SEQ ID NO:7, SEQ ID NO:8, SEQ ID NO:10, SEQ ID NO:12, SEQ ID NO:14, SEQ ID NO:16, SEQ ID NO:18, or SEQ ID NO:20.

54. The composition of claim 45, wherein the enzyme breaker comprises the amino acid sequence of SEQ ID NO:2.

55. The composition of claim 45, wherein the enzyme breaker is encoded by a polynucleotide having the nucleic acids sequence of SEQ ID NO:1.

56. The composition of claim 45, wherein the enzyme breaker is a cellulase encoded by the nucleic acid sequence of SEQ ID NO:3.

57. The composition of claim 53, wherein the mutated variant cellulase is expressed at least 1.0 g/L, 2.0 g/L, 3.0 g/L, 4.0 g/L, 5.0 g/L, 6.0 g/L, 7.0 g/L, 8.0 g/L, 9.0 g/L, 10.0 g/L, 11.0 g/L, 12.0 g/L, 13.0 g/L, 14.0 g/L, 15.0 g/L, 16.0 g/L, 17.0 g/L, 18.0 g/L, 19.0 g/L, 20.0 g/L, 21.0 g/L, 22.0 g/L, 23.0 g/L, 24.0 g/L, 25.0, g/L, 26.0 g/L, 27.0 g/L, 28.0 g/L, 29.0 g/L, 30.0 g/L, 31.0 g/L, 32.0 g/L, 33.0 g/L, 34.0 g/L, or 35.0 g/L.

58. The composition of claim 45, wherein the enzyme breaker is a mutated variant of the wild-type cellulase, and has a melting temperature that is at least 20° F. greater than the melting temperature of the wild-type cellulase at about pH 6.5 and at least 10° F. greater than the melting temperature of the wild-type cellulase at about pH 10.5.

59. A method of reducing the viscosity of a polysaccharide gel comprising β-1,4 glycosidic bonds, the method comprising contacting the polysaccharide gel with a cellulase variant under permissive conditions for a period of time sufficient to allow the cellulase variant to hydrolyze the β-1,4 glycosidic bonds in the polysaccharide gel thereby reducing the viscosity of the polysaccharide gel, wherein the cellulase variant is derived from a hyperthermophilic bacterium, and wherein the cellulase variant exhibits increased temperature and pH tolerance compared to the wild-type cellulase.

60. The method of claim 59, wherein the polysaccharide gel comprises a linear guar, a crosslinked guar, or mixtures thereof.

61. The method of claim 59, wherein the permissive conditions comprise (a) a temperature range of from about 180° F. to about 275° F., (b) a pH range of from about 9 to about 11, or both.

62. The method of claim 59, wherein the cellulase variant is:
(a) a mutated cellulase variant comprising 12 mutations relative to the wild type cellulase;
(b) a mutated cellulase comprising at least one mutation selected from F38Y, Y61Q, M69E, D70P, R71S, 194Q, I166V, S183R, S191A, E212P, L231V, M276A, E277S, R280G, T297P, T301Q, and any combination thereof;
(c) a cellulase derived from Thermotoga maritima comprising at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(d) a cellulase encoded by a nucleotide sequence of SEQ ID NO:3 comprising at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(e) a cellulase encoded by a nucleotide sequence from Thermotoga maritima having at least one mutation which increases the expression level of a protein encoded by said nucleotide sequence compared to a Thermotoga maritima genomic sequence;
(f) a cellulase encoded by a first nucleotide sequence encoding the polypeptide of SEQ ID NO:2 wherein said first nucleotide sequence has been mutated with respect to a second sequence encoding SEQ ID NO:2 such that the expression level of said cellulase is increased relative to that of a protein encoded by said second nucleotide sequence;
(g) a cellulase encoded by a nucleotide sequence encoding a protein at least 50%, 60%, 70%, 80%, 90%, 95%, 97%, 98%, 99% or 100% identical to SEQ ID NO:2, or a fragment thereof, wherein said nucleotide sequence comprises at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(h) a cellulase comprising the amino acid sequence of SEQ ID NO:2, SEQ ID NO:6, SEQ ID NO:9, SEQ ID NO:11, SEQ ID NO:13, SEQ ID NO:15, SEQ ID NO:17, SEQ ID NO:19, or SEQ ID NO:21; or
(i) a cellulase encoded by the nucleic acid sequence of SEQ ID NO:1, SEQ ID NO:3, SEQ ID NO:4, SEQ ID NO:5, SEQ ID NO:7, SEQ ID NO:8, SEQ ID NO:10, SEQ ID NO:12, SEQ ID NO:14, SEQ ID NO:16, SEQ ID NO:18, or SEQ ID NO:20.

63. A method of treating a subterranean formation, comprising: introducing into the subterranean formation a fracturing fluid that comprises a polysaccharide gel and a cellulase variant; and allowing the polysaccharide gel and the cellulase variant to react under permissive conditions for a period of time sufficient to allow the cellulase variant to hydrolyze at least some of the β-1,4 glycosidic bonds, but not the α-1,6 glycosidic bonds, in the polysaccharide gel, thereby generating a reduced viscosity fracturing fluid; wherein the cellulase variant is derived from a hyperthermophilic bacterium, and wherein the cellulase variant exhibits increased temperature and pH tolerance compared to the wild-type cellulase.

64. The method of claim 63, wherein the reduced viscosity fracturing fluid generated by the cellulase variant comprises less residue that a comparable reduced viscosity fracturing fluid generated by a chemical breaker, wherein the dosage of cellulase variant and chemical breaker provide substantially the same reduction in viscosity.

65. The method of claim 63, wherein the cellulase variant is:
(a) a mutated cellulase variant comprising 12 mutations relative to the wild type cellulase;
(b) a mutated cellulase comprising at least one mutation selected from F38Y, Y61Q, M69E, D70P, R71S, 194Q, I166V, S183R, S191A, E212P, L231V, M276A, E277S, R280G, T297P, T301Q, and any combination thereof;
(c) a cellulase derived from Thermotoga maritima comprising at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(d) a cellulase encoded by a nucleotide sequence of SEQ ID NO:3 comprising at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(e) a cellulase encoded by a nucleotide sequence from Thermotoga maritima having at least one mutation which increases the expression level of a protein encoded by said nucleotide sequence compared to a Thermotoga maritima genomic sequence;
(f) a cellulase encoded by a first nucleotide sequence encoding the polypeptide of SEQ ID NO:2 wherein said first nucleotide sequence has been mutated with respect to a second sequence encoding SEQ ID NO:2 such that the expression level of said cellulase is increased relative to that of a protein encoded by said second nucleotide sequence;
(g) a cellulase encoded by a nucleotide sequence encoding a protein at least 50%, 60%, 70%, 80%, 90%, 95%, 97%, 98%, 99% or 100% identical to SEQ ID NO:2, or a fragment thereof, wherein said nucleotide sequence comprises at least one mutation selected from T6C, T9C, T15G, A22C, G24T, A33C, A39C, A40C, A42C, A54C, A57C, T66C, G81A, A84C, A6C, G6C, A9C, G9C, A15G, C15G, T22C, G22C, A24T, C24T, T33C, G33C, T39C, G39C, T40C, G40C, T42C, G42C, T54C, G54C, T57C, G57C, A66C, G66C, C81A, T81A, T84C, G84C, and any combination thereof;
(h) a cellulase comprising the amino acid sequence of SEQ ID NO:2, SEQ ID NO: 6, SEQ ID NO:9, SEQ ID NO:11, SEQ ID NO:13, SEQ ID NO:15, SEQ ID NO:17, SEQ ID NO:19, or SEQ ID NO:21; or
(i) a cellulase encoded by the nucleic acid sequence of SEQ ID NO:1, SEQ ID NO:3, SEQ ID NO:4, SEQ ID NO:5, SEQ ID NO:7, SEQ ID NO:8, SEQ ID NO:10, SEQ ID NO:12, SEQ ID NO:14, SEQ ID NO:16, SEQ ID NO:18, or SEQ ID NO:20.


SEQUENCE LISTING (21 sequences)

SEQ ID NO: 1 - DNA, 954 bp, Artificial Sequence
  Open reading frame encoding SEQ ID NO2 conveying enhanced protein expression
atgggcgtcgatccgtttgaacgtaacaaaatcttgggccgcggcattaatatcggcaatgcgctcgaagcaccaaatgaaggcgactggggagtggtgataaaagatgagttcttcgacattataaaagaagccggtttctctcatgttcgaattccaataagatggagtacgcacgctcaggcgtttcctccttataaaatcgagccttctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaatga

SEQ ID NO: 2 - protein, 317 aa, Thermotoga maritima
MGVDPFERNKILGRGINIGNALEAPNEGDWGVVIKDEFFDIIKEAGFSHVRIPIRWSTHAQAFPPYKIEPSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHLIEEFNFIEEWSKKNKRPIYIGEFGAYRKADLESRIKWTSFVVREAEKRGWSWAYWEFCSGFGVYDPLRKQWNKDLLEALIGGDSIE

SEQ ID NO: 3 - DNA, 954 bp, Artificial Sequence
  Open reading frame encoding SEQ ID NO2
atgggtgttgatccttttgaaaggaacaaaatattgggaagaggcattaatataggaaatgcgcttgaagcaccaaatgagggagactggggagtggtgataaaagatgagttcttcgacattataaaagaagccggtttctctcatgttcgaattccaataagatggagtacgcacgctcaggcgtttcctccttataaaatcgagccttctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaataa

SEQ ID NO: 4 - DNA, 976 bp, Artificial Sequence
  5′ region and open reading frame encoding SEQ ID NO2 having modest increased expression
tctactagttaggaggtaacttatgggcgtcgatccgtttgaacgtaacaaaatcttgggccgcggcattaatatcggcaatgcgctcgaagcaccaaatgaaggcgactggggagtggtgataaaagatgagttcttcgacattataaaagaagccggtttctctcatgttcgaattccaataagatggagtacgcacgctcaggcgtttcctccttataaaatcgagccttctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaatga

SEQ ID NO: 5 - DNA, 954 bp, Thermotoga maritima
atgggtgttgatccttttgaaaggaacaaaatattgggaagaggcattaatataggaaatgcgcttgaagcaccaaatgagggagactggggagtggtgataaaagatgagttcttcgacattataaaagaagccggtttctctcatgttcgaattccaataagatggagtacgcacgcttacgcgtttcctccttataaaatcatggatcgcttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactataattataggcacagctgaatgggggggtatatctgcccttgaaaaactgtctgtcccaaaatgggaaaaaaattctatagttacaattcactactacaatcctttcgaatttacccatcaaggagctgagtgggtggaaggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaaatggagaaaaggagatggagctgggcatactgggaattttgttccggttttggtgtttatgatactctgagaaaaacctggaataaagatcttttagaagctttaataggaggagatagcattgaataa

SEQ ID NO: 6 - protein, 317 aa, Thermotoga maritima [sequence omitted]

SEQ ID NO: 7 - DNA, 954 bp, Artificial Sequence
  Synthetically generated polynucleotide
atgggtgttgatccttttgaaaggaacaaaatattgggaagaggcattaatataggaaatgcgcttgaagcaccaaatgagggcgactggggagtcgtgataaaagatgagttcttcgacattataaaagaagccggtttctctcatgttcgaattccaataagatggagtacgcacgcttacgcgtttcctccttataaaatcatggatcgcttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactataattataggcacagctgaatgggggggtatatctgcccttgaaaaactgtctgtcccaaaatgggaaaaaaattctatagttacaattcactactacaatcctttcgaatttacccatcaaggagctgagtgggtggaaggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaaatggagaaaaggagatggagctgggcatactgggaattttgttccggttttggtgtttatgatactctgagaaaaacctggaataaagatcttttagaagctttaataggaggagatagcattgaataa

SEQ ID NO: 8 - DNA, 974 bp, Artificial Sequence
  Synthetically generated polynucleotide
atgggtgttgatccttttgaaaggaacaaaatattgggaagaggcattaatataggaaatgcgcttgaagcaccaaatgagggagactggggagtggtgataaaagatgagtatttcgacattataaaagaagccggtttctctcatgttcgaattccaataagatggagtacgcacgctcaggcgtttcctccttataaaatcgaggatcgcttcttcaaaagagtggatgaagtgataaacggagccctgaaaagaggactggctgttgttataaatcagcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactataattataggcacagctgaatgggggggtatatctgcccttgaaaaactgagggtcccaaaatgggaaaaaaatgcgatagttacaattcactactacaatcctttcgaatttacccatcaaggagctgagtgggtggaaggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagctgagaaaaggagatggagctgggcatactgggaattttgttccggttttggtgtttatgatactctgagaaaaacctggaataaagatcttttagaagctttaataggaggagatagcattgaataacaccattccaagatggcgtg

SEQ ID NO: 9 - protein, 320 aa, Artificial Sequence
  Synthetically generated polypeptide
MGVDPFERNKILGRGINIGNALEAPNEGDWGVVIKDEYFDIIKEAGFSHVRIPIRWSTHAQAFPPYKIEDRFFKRVDEVINGALKRGLAVVINQHHYEELMNDPEEHKERFLALWKQIADRYKDYPETLFFEILNEPHGNLTPEKWNELLEEALKVIRSIDKKHTIIIGTAEWGGISALEKLRVPKWEKNAIVTIHYYNPFEFTHQGAEWVEGSEKWLGRKWGSPDDQKHLIEEFNFIEEWSKKNKRPIYIGEFGAYRKADLESRIKWTSFVVREAEKRRWSWAYWEFCSGFGVYDTLRKTWNKDLLEALIGGDSIEHHS

SEQ ID NO: 10 - DNA, 954 bp, Artificial Sequence
  Synthetically generated polynucleotide
atgggtgttgatccttttgaaaggaacaaaatattgggaagaggcattaatataggaaatgcgcttgaagcaccaaatgagggagactggggagtggtgataaaagatgagtatttcgacattataaaagaagccggtttctctcatgttcgaattccaataagatggagtacgcacgctcaggcgtttcctccttataaaatcgaggattctttcttcaaaagagtggatgaagtgataaacggagccctgaaaagaggactggctgttgttataaatattcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatgtgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctctaataggaggagatagcattgaataa

SEQ ID NO: 11 - protein, 317 aa, Artificial Sequence
  Synthetically generated polypeptide
MGVDPFERNKILGRGINIGNALEAPNEGDWGVVIKDEYFDIIKEAGFSHVRIPIRWSTHAQAFPPYKIEDSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHVIEEFNFIEEWSKKNKRPIYIGEFGAYRKADLESRIKWTSFVVREAEKRGWSWAYWEFCSGFGVYDPLRKQWNKDLLEALIGGDSIE

SEQ ID NO: 12 - DNA, 954 bp, Artificial Sequence
  Synthetically generated polynucleotide
atgggtgttgatccttttgaaaggaacaaaatattgggaagaggcattaatataggaaatgcgcttgaagcaccaaatgagggagactggggagtggtgataaaagatgagttcttcgacattataaaagaagccggtttctctcatgttcgaattccaataagatggagtacgcacgctcaggcgtttcctccttataaaatcgaggattctttcttcaaaagagtggatgaagtgataaacggagccctgaaaagaggactggctgttgttataaatcagcatcactacgaggagttaatgaatgatccagaagaacacaaggaaagatttcttgctctttggaaacaaattgctgatcgttataaagactatcccgaaactctattttttgaaattctgaatgaacctcacggaaatcttactccggaaaaatggaatgaactgcttgaggaagctctaaaagttataagatcaattgacaaaaagcacactgtgattataggcacagctgaatgggggggtatatctgcccttgaaaaactgagggtcccaaaatgggaaaaaaatgcgatagttacaattcactactacaatcctttcgaatttacccatcaaggagctgagtgggtgcctggatctgagaaatggttgggaagaaagtggggatctccagatgatcagaaacatttgatagaagaattcaattttatagaagaatggtcaaaaaagaacaaaagaccaatttacataggtgagtttggtgcctacagaaaagctgaccttgaatcaagaataaaatggacctcctttgtcgttcgcgaagccgagaaaagggggtggagctgggcatactgggaattttgttccggttttggtgtttatgatcctctgagaaaacagtggaataaagatcttttagaagctttaataggaggagatagcattgaataa

SEQ ID NO: 13 - protein, 317 aa, Artificial Sequence
  Synthetically generated polypeptide
MGVDPFERNKILGRGINIGNALEAPNEGDWGVVIKDEFFDIIKEAGFSHVRIPIRWSTHAQAFPPYKIEDSFFKRVDEVINGALKRGLAVVINQHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHLIEEFNFIEEWSKKNKRPIYIGEFGAYRKADLESRIKWTSFVVREAEKRGWSWAYWEFCSGFGVYDPLRKQWNKDLLEALIGGDSIE

SEQ ID NO: 14 - DNA, 972 bp, Thermotoga sp.
atggaacagtcagttgctgaaagtgatagcaactcagcatttgaatacaacaaaatggtaggtaaaggagtaaatattggaaatgctttagaagctcctttcgaaggagcttggggagtaagaattgaggatgaatattttgagataataaagaaaaggggatttgattctgttaggattcccataagatggtcagcacatatatccgaaaagccaccatatgatattgacaggaatttcctcgaaagagttaaccatgttgtcgatagggctcttgagaataatttaacagtaatcatcaatacgcaccattttgaagaactctatcaagaaccggataaatacggcgatgttttggtggaaatttggagacagattgcaaaattctttaaagattacccggaaaatctgttctttgaaatctacaacgagcctgctcagaacttgacagctgaaaaatggaacgcactttatccaaaagtgctcaaagttatcagggagagcaatccaacccggattgtcattatcgatgctccaaactgggcacactatagcgcagtgagaagtctaaaattagtcaacgacaaacgcatcattgtttccttccattactacgaacctttcaaattcacacatcagggtgccgaatgggttaatcccatcccacctgttagggttaagtggaatggcgaggaatgggaaattaaccaaatcagaagtcatttcaaatacgtgagtgactgggcaaagcaaaataacgtaccaatctttcttggtgaattcggtgcttattcaaaagcagacatggactcaagggttaagtggaccgaaagtgtgagaaaaatggcggaagaatttggattttcatacgcgtattgggaattttgtgcaggatttggcatatacgatagatggtctcaaaactggatcgaaccattggcaacagctgtggttggcacaggcaaagagtaa

SEQ ID NO: 15 - protein, 323 aa, Thermotoga sp.
MEQSVAESDSNSAFEYNKMVGKGVNIGNALEAPFEGAWGVRIEDEYFEIIKKRGFDSVRIPIRWSAHISEKPPYDIDRNFLERVNHVVDRALENNLTVIINTHHFEELYQEPDKYGDVLVEIWRQIAKFFKDYPENLFFEIYNEPAQNLTAEKWNALYPKVLKVIRESNPTRIVIIDAPNWAHYSAVRSLKLVNDKRIIVSFHYYEPFKFTHQGAEWVNPIPPVRVKWNGEEWEINQIRSHFKYVSDWAKQNNVPIFLGEFGAYSKADMDSRVKWTESVRKMAEEFGFSYAYWEFCAGFGIYDRWSQNWIEPLATAVVGTGKE

SEQ ID NO: 16 - DNA, 1042 bp, Artificial Sequence
  Synthetically generated polynucleotide
ggatccaccatgagggtgttgctcgttgccctcgctctcctggctctcgctgcgagcgccaccagcggcgtggacccgttcgagaggaacaagatcctgggcaggggcatcaacatcggcaacgccctggaggccccgaacgagggcgactggggcgtggtgatcaaggacgagtacttcgacatcatcaaggaggccggcttcagccacgtgagaatcccgatcaggtggagcacccacgcccaggccttcccgccgtacaagatcgaggacaggttcttcaagagggtggacgaggtgatcaacggcgccctgaagaggggcctggccgtggtgatcaaccagcaccactacgaggagctgatgaacgacccggaggagcacaaggagaggttcctggccctgtggaagcagatcgccgacaggtacaaggactacccggagaccctgttcttcgagatcctgaacgagccgcacggcaacctgaccccggagaagtggaacgagctgctggaggaggccctgaaggtgatcaggagcatcgacaagaagcacaccatcatcatcggcaccgccgagtggggcggcatcagcgccctggagaagctgagggtgccgaagtgggagaagaacgccatcgtgaccatccactactacaacccgttcgagttcacccaccagggcgccgagtgggtggagggcagcgagaagtggctgggcaggaagtggggcagcccggacgaccagaagcacctgatcgaggagttcaacttcatcgaggagtggagcaagaagaacaagaggccgatctacatcggcgagttcggcgcctacaggaaggccgacctggagagcaggatcaagtggaccagcttcgtggtgagggaggccgagaagaggaggtggagctgggcctactgggagttctgcagcggcttcggcgtgtacgacaccctgaggaagacctggaacaaggacctgctggaggccctgatcggcggcgacagcatcgagagcgagaaggacgagctgtgagagctca

SEQ ID NO: 17 - protein, 341 aa, Artificial Sequence
  Synthetically generated polypeptide
MRVLLVALALLALAASATSGVDPFERNKILGRGINIGNALEAPNEGDWGVVIKDEYFDIIKEAGFSHVRIPIRWSTHAQAFPPYKIEDRFFKRVDEVINGALKRGLAVVINQHHYEELMNDPEEHKERFLALWKQIADRYKDYPETLFFEILNEPHGNLTPEKWNELLEEALKVIRSIDKKHTIIIGTAEWGGISALEKLRVPKWEKNAIVTIHYYNPFEFTHQGAEWVEGSEKWLGRKWGSPDDQKHLIEEFNFIEEWSKKNKRPIYIGEFGAYRKADLESRIKWTSFVVREAEKRRWSWAYWEFCSGFGVYDTLRKTWNKDLLEALIGGDSIESEKDEL

SEQ ID NO: 18 - DNA, 1042 bp, Artificial Sequence
  Synthetically generated polynucleotide
ggatccaccatgagggtgttgctcgttgccctcgctctcctggctctcgctgcgagcgccaccagcggcgtggacccgttcgagaggaacaagatcctgggcaggggcatcaacatcggcaacgccctggaggccccgaacgagggcgactggggcgtggtgatcaaggacgagttcttcgacatcatcaaggaggccggcttcagccacgtgagaatcccgatcaggtggagcacccacgcccaggccttcccgccgtacaagatcgagccgagcttcttcaagagggtggacgaggtgatcaacggcgccctgaagaggggcctggccgtggtgatcaacatccaccactacgaggagctgatgaacgacccggaggagcacaaggagaggttcctggccctgtggaagcagatcgccgacaggtacaaggactacccggagaccctgttcttcgagatcctgaacgagccgcacggcaacctgaccccggagaagtggaacgagctgctggaggaggccctgaaggtgatcaggagcatcgacaagaagcacaccgtgatcatcggcaccgccgagtggggcggcatcagcgccctggagaagctgagggtgccgaagtgggagaagaacgccatcgtgaccatccactactacaacccgttcgagttcacccaccagggcgccgagtgggtgccgggcagcgagaagtggctgggcaggaagtggggcagcccggacgaccagaagcacctgatcgaggagttcaacttcatcgaggagtggagcaagaagaacaagaggccgatctacatcggcgagttcggcgcctacaggaaggccgacctggagagcaggatcaagtggaccagcttcgtggtgagggaggccgagaagaggggctggagctgggcctactgggagttctgcagcggcttcggcgtgtacgacccgctgaggaagcagtggaacaaggacctgctggaggccctgatcggcggcgacagcatcgagagcgagaaggacgagctgtgagagctca

SEQ ID NO: 19 - protein, 341 aa, Artificial Sequence
  Synthetically generated polypeptide
MRVLLVALALLALAASATSGVDPFERNKILGRGINIGNALEAPNEGDWGVVIKDEFFDIIKEAGFSHVRIPIRWSTHAQAFPPYKIEPSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHLIEEFNFIEEWSKKNKRPIYIGEFGAYRKADLESRIKWTSFVVREAEKRGWSWAYWEFCSGFGVYDPLRKQWNKDLLEALIGGDSIESEKDEL

SEQ ID NO: 20 - DNA, 1042 bp, Artificial Sequence
  Synthetically generated polynucleotide
ggatccaccatgagggtgttgctcgttgccctcgctctcctggctctcgctgcgagcgccaccagcggcgtggacccgttcgagaggaacaagatcctgggcaggggcatcaacatcggcaacgccctggaggccccgaacgagggcgactggggcgtggtgatcaaggacgagtacttcgacatcatcaaggaggccggcttcagccacgtgagaatcccgatcaggtggagcacccacgcccaggccttcccgccgtacaagatcgaggacagcttcttcaagagggtggacgaggtgatcaacggcgccctgaagaggggcctggccgtggtgatcaacatccaccactacgaggagctgatgaacgacccggaggagcacaaggagaggttcctggccctgtggaagcagatcgccgacaggtacaaggactacccggagaccctgttcttcgagatcctgaacgagccgcacggcaacctgaccccggagaagtggaacgagctgctggaggaggccctgaaggtgatcaggagcatcgacaagaagcacaccgtgatcatcggcaccgccgagtggggcggcatcagcgccctggagaagctgagggtgccgaagtgggagaagaacgccatcgtgaccatccactactacaacccgttcgagttcacccaccagggcgccgagtgggtgccgggcagcgagaagtggctgggcaggaagtggggcagcccggacgaccagaagcacgtgatcgaggagttcaacttcatcgaggagtggagcaagaagaacaagaggccgatctacatcggcgagttcggcgcctacaggaaggccgacctggagagcaggatcaagtggaccagcttcgtggtgagggaggccgagaagaggggctggagctgggcctactgggagttctgcagcggcttcggcgtgtacgacccgctgaggaagcagtggaacaaggacctgctggaggccctgatcggcggcgacagcatcgagagcgagaaggacgagctgtgagagctca

SEQ ID NO: 21 - protein, 341 aa, Artificial Sequence
  Synthetically generated polypeptide
MRVLLVALALLALAASATSGVDPFERNKILGRGINIGNALEAPNEGDWGVVIKDEYFDIIKEAGFSHVRIPIRWSTHAQAFPPYKIEDSFFKRVDEVINGALKRGLAVVINIHHYEELMNDPEEHKERFLALWKQIADRYKDYPETLFFEILNEPHGNLTPEKWNELLEEALKVIRSIDKKHTVIIGTAEWGGISALEKLRVPKWEKNAIVTIHYYNPFEFTHQGAEWVPGSEKWLGRKWGSPDDQKHVIEEFNFIEEWSKKNKRPIYIGEFGAYRKADLESRIKWTSFVVREAEKRGWSWAYWEFCSGFGVYDPLRKQWNKDLLEALIGGDSIESEKDEL